SEQUENCE LISTING (47 sequences)

SEQ ID NO: 1 - protein, 10 aa, Artificial Sequence
  Mefp1
AKPSYPPTYK

SEQ ID NO: 2 - DNA, 30 bp, Artificial Sequence
  sense primer
tacaaagctaagccgtcttatccgccaacc

SEQ ID NO: 3 - DNA, 30 bp, Artificial Sequence
  antisense primer
tttgtaggttggcggataagacggcttagc

SEQ ID NO: 4 - DNA, 17 bp, Artificial Sequence
  sense primer
gatccgaattccccggg

SEQ ID NO: 5 - DNA, 19 bp, Artificial Sequence
  antisense primer
tttgtacccggggaattcg

SEQ ID NO: 6 - DNA, 22 bp, Artificial Sequence
  sense primer
tacaaacgtaagcttgtcgacc

SEQ ID NO: 7 - DNA, 20 bp, Artificial Sequence
  antisense primer
tcgaggtcgacaagcttacg

SEQ ID NO: 8 - DNA, 36 bp, Artificial Sequence
  pET-22b(+)OmpASP1-3-7XMefp1* sense primer
catatgaaaaaggctaagccgtcttatccgccaacc

SEQ ID NO: 9 - DNA, 39 bp, Artificial Sequence
  pET22b(+)OmpASP1-4-7XMefp1* sense primer
catatgaaaaagacagctaagccgtcttatccgccaacc

SEQ ID NO: 10 - DNA, 42 bp, Artificial Sequence
  pET22b(+)OmpASP1-5-7XMefp1* sense primer
catatgaaaaagacagctgctaagccgtcttatccgccaacc

SEQ ID NO: 11 - DNA, 45 bp, Artificial Sequence
  pET22b(+)OmpASP1-6-7XMefp1* sense primer
catatgaaaaagacagctatcgctaagccgtcttatccgccaacc

SEQ ID NO: 12 - DNA, 48 bp, Artificial Sequence
  pET22b(+)OmpASP1-7-7XMefp1* sense primer
catatgaaaaagacagctatcgcggctaagccgtcttatccgccaacc

SEQ ID NO: 13 - DNA, 51 bp, Artificial Sequence
  pET22b(+)-OmpASP1-8-7XMefp1* sense primer
catatgaaaaagacagctatcgcgattgctaagccgtcttatccgccaacc

SEQ ID NO: 14 - DNA, 54 bp, Artificial Sequence
  pET22b(+)OmpASP1-9-7XMefp1* sense primer
catatgaaaaagacagctatcgcgattgcagctaagccgtcttatccgccaacc

SEQ ID NO: 15 - DNA, 57 bp, Artificial Sequence
  pET22b(+)OmpASP1-10-7XMefp1* sense primer
catatgaaaaagacagctatcgcgattgcagtggctaagccgtcttatccgccaacc

SEQ ID NO: 16 - DNA, 60 bp, Artificial Sequence
  pET22b(+)OmpASP1-11-7XMefp1* sense primer
catatgaaaaagacagctatcgcgattgcagtggcagctaagccgtcttatccgccaacc

SEQ ID NO: 17 - DNA, 66 bp, Artificial Sequence
  pET22b(+)OmpASP1-13-7XMefp1* sense primer
catatgaaaaagacagctatcgcgattgcagtggcactggctgctaagccgtcttatccgccaacc

SEQ ID NO: 18 - DNA, 72 bp, Artificial Sequence
  pET22b(+)OmpASP1-15-7XMefp1* sense primer
catatgaaaaagacagctatcgcgattgcagtggcactggctggtttcgctaagccgtcttatccgccaacc

SEQ ID NO: 19 - DNA, 90 bp, Artificial Sequence
  pET22b(+)OmpASP1-21-7XMefp1* sense primer
catatgaaaaagacagctatcgcgattgcagtggcactggctggtttcgctaccgtagcgcaggccgctaagccgtcttatccgccaacc

SEQ ID NO: 20 - DNA, 96 bp, Artificial Sequence
  pET22b(+)OmpASP1-23-7XMefp1* sense primer
catatgaaaaagacagctatcgcgattgcagtggcactggctggtttcgctaccgtagcgcaggccgctccggctaagccgtcttatccgccaacc

SEQ ID NO: 21 - DNA, 63 bp, Artificial Sequence
  pET22b(+)OmpASP1-8-Xa-7XMefp1* sense primer
catatgaaaaagacagctatcgcgattatcgaaggtcgtgctaagccgtcttatccgccaacc

SEQ ID NO: 22 - DNA, 69 bp, Artificial Sequence
  pET22b(+)OmpASP1-8-SmaI-Xa-7XMefp1* sense primer
catatgaaaaagacagctatcgcgattcccgggatcgaaggtcgtgctaagccgtcttatccgccaacc

SEQ ID NO: 23 - DNA, 21 bp, Artificial Sequence
  reverse primer
ctcgaggtcgacaagcttacg

SEQ ID NO: 24 - DNA, 39 bp, Artificial Sequence
  pET22b(+)OmpASP1-4 ofHepI** sense primer
catatgaaaaagacacacatcagccacatctccatgtgc

SEQ ID NO: 25 - DNA, 45 bp, Artificial Sequence
  pET22b(+)OmpASP1-6 ofHepI** sense primer
catatgaaaaagacagctatccacatcagccacatctccatgtgc

SEQ ID NO: 26 - DNA, 51 bp, Artificial Sequence
  pET22b(+)OmpASP1-8 ofHepI** snse primer
catatgaaaaagacagctatcgcgattcacatcagccacatctccatgtgc

SEQ ID NO: 27 - DNA, 57 bp, Artificial Sequence
  pET22b(+)OmpASP1-10 ofHepI** primer
catatgaaaaagacagctatcgcgattgcagtgcacatcagccacatctccatgtgc

SEQ ID NO: 28 - DNA, 63 bp, Artificial Sequence
  pET22b(+)OmpASP1-12 ofHepI** sense primer
catatgaaaaagacagctatcgcgattgcagtggcactgcacatcagccacatctccatgtgc

SEQ ID NO: 29 - DNA, 87 bp, Artificial Sequence
  pET22b(+)OmpASP1-10-6XTrp-Xa-ofHepI** sense primer
catatgaaaaagacagctatcgcgattgcagtgtggtggtggtggtggtggatcgaaggtcgtcacatcagccacatctccatgtgc

SEQ ID NO: 30 - DNA, 87 bp, Artificial Sequence
  pET22b(+)OmpASP1-10-6XArg-Xa-ofHepI** sense primer
catatgaaaaagacagctatcgcgattgcagtgcgccgtcgccgtcgccgtatcgaaggtcgtcacatcagccacatctccatgtgc

SEQ ID NO: 31 - DNA, 87 bp, Artificial Sequence
  pET22b(+)OmpASP1-10-6XLys-Xa-ofHepI** sense primer
catatgaaaaagacagctatcgcgattgcagtgaaaaagaaaaagaaaaagatcgaaggtcgtcacatcagccacatctccatgtgc

SEQ ID NO: 32 - DNA, 87 bp, Artificial Sequence
  pET22b(+)OmpASP1-10-6XGlu-Xa-ofHepI** sense primer
catatgaaaaagacagctatcgcgattgcagtggaagaggaagaggaagagatcgaaggtcgtcacatcagccacatctccatgtgc

SEQ ID NO: 33 - DNA, 87 bp, Artificial Sequence
  pET22b(+)OmpASP1-10-6XAsp-Xa-ofHepI** sense primer
catatgaaaaagacagctatcgcgattgcagtggacgatgacgatgacgatatcgaaggtcgtcacatcagccacatctccatgtgc

SEQ ID NO: 34 - DNA, 87 bp, Artificial Sequence
  pET22b(+)OmpASP1-10-6XTyr-Xa-ofHepI** sense primer
catatgaaaaagacagctatcgcgattgcagtgtactattactattactatatcgaaggtcgtcacatcagccacatctccatgtgc

SEQ ID NO: 35 - DNA, 87 bp, Artificial Sequence
  pET22b(+)OmpASP1-10-6XPhe-Xa-ofHepI** sense primer
catatgaaaaagacagctatcgcgattgcagtgttctttttctttttctttatcgaaggtcgtcacatcagccacatctccatgtgc

SEQ ID NO: 36 - DNA, 75 bp, Artificial Sequence
  pET22b(+)OmpASP1-6-6XArg-Xa-ofHepI** sense primer
catatgaaaaagacagctatccgccgtcgccgtcgccgtatcgaaggtcgtcacatcagccacatctccatgtgc

SEQ ID NO: 37 - DNA, 81 bp, Artificial Sequence
  pET22b(+)OmpASP1-8-6XArg-Xa-ofHepI** sense primer
catatgaaaaagacagctatcgcgattcgccgtcgccgtcgccgtatcgaaggtcgtcacatcagccacatctccatgtgc

SEQ ID NO: 38 - DNA, 93 bp, Artificial Sequence
  pET22b(+)OmpASP1-12-6XArg-Xa-ofHepI** sense primer
catatgaaaaagacagctatcgcgattgcagtggcactgcgccgtcgccgtcgccgtatcgaaggtcgtcacatcagccacatctccatgtgc

SEQ ID NO: 39 - DNA, 99 bp, Artificial Sequence
  pET22b(+)OmpASP1-14-6XArg-Xa-ofHepI* sense primer
catatgaaaaagacagctatcgcgattgcagtggcactggctggtcgccgtcgccgtcgccgtatcgaaggtcgtcacatcagccacatctccatgtgc

SEQ ID NO: 40 - DNA, 69 bp, Artificial Sequence
  pET22b(+)OmpASP1-10-Xa-ofHepI** sense primer
catatgaaaaagacagctatcgcgattgcagtgatcgaaggtcgtcacatcagccacatctccatgtgc

SEQ ID NO: 41 - DNA, 75 bp, Artificial Sequence
  pET22b(+)OmpASP1-10-LysArg-Xa-ofHepI** sense primer
catatgaaaaagacagctatcgcgattgcagtgaaacgcatcgaaggtcgtcacatcagccacatctccatgtgc

SEQ ID NO: 42 - DNA, 81 bp, Artificial Sequence
  pET22b(+)OmpASP1-10-4XArg-Xa-ofHepI** sense primer
catatgaaaaagacagctatcgcgattgcagtgcgccgtcgccgtatcgaaggtcgtcacatcagccacatctccatgtgc

SEQ ID NO: 43 - DNA, 93 bp, Artificial Sequence
  pET22b(+)OmpASP1-10-8XArg-Xa-ofHepI** sense primer
catatgaaaaagacagctatcgcgattgcagtgcgccgtcgccgtcgccgtcgccgtatcgaaggtcgtcacatcagccacatctccatgtgc

SEQ ID NO: 44 - DNA, 99 bp, Artificial Sequence
  pET22b(+)OmpASP1-10-10XArg-Xa-ofHepI** sense primer
catatgaaaaagacagctatcgcgattgcagtgcgccgtcgccgtcgccgtcgccgtcgccgtatcgaaggtcgtcacatcagccacatctccatgtgc

SEQ ID NO: 45 - DNA, 45 bp, Artificial Sequence
  reverse primer
ctcgaggtcgacaagcttttcgaacttgcagcaggggccacagcc

SEQ ID NO: 46 - protein, 23 aa, Artificial Sequence
  OmpA signal peptide
MKKTAIAIAVALAGFATVAQAAP

SEQ ID NO: 47 - protein, 26 aa, Paralichthys olivaceus
HISHISMCRWCCNCCKAKGCGPCCKF

(19) United States
(12) Patent Application Publication
(10) Pub. No.: US 2009/0011995 A1
(43) Pub. Date: Jan. 8, 2009

(54) PRODUCTION OF A SOLUBLE NATIVE FORM OF RECOMBINANT PROTEIN BY THE SIGNAL SEQUENCE AND SECRETIONAL ENHANCER

(76) Inventors: Sang Jun Lee, Busan (KR); Young Ok Kim, Busan (KR); Bo-Hye Nam, Busan (KR)

Correspondence Address: KLARQUIST SPARKMAN, LLP, 121 SW SALMON STREET, SUITE 1600, PORTLAND, OR, 97204, US

(21) Appl. No.: 12/162,118

(22) PCT Filed: Jan. 30, 2007
(86) PCT No.: PCT/KR07/00515

§ 371 Date: Jul. 24, 2008

(30) Foreign Application Priority Data
Jan. 31, 2006 (KR) 10-2006-0009418
Mar. 9, 2006 (KR) 10-2006-0022389

(51) Int. Cl. A61K 38/16; C12N 15/64; C12N 1/21; C12P 21/00; C12N 5/10; A61P 25/00; C12N 1/19; C12Q 1/68; C07K 14/00

(52) U.S. Cl. 514/12; 435/320.1; 435/252.33; 435/69.7; 435/348; 435/410; 435/254.2; 435/6; 530/412

(57) ABSTRACT

The present invention is drawn to a method for enhancing secretional efficiency of a heterologous protein using a secretional enhancer consisting of a modified signal sequence which comprises the N-region of a signal sequence and/or a hydrophobic fragment of the said signal sequence comprising the said N-region and/or the hydrophilic polypeptide. The method of the present invention can be used not only for production of recombinant heterologous proteins by inhibiting insoluble precipitation and enhancing secretional efficiency of the recombinant protein into the periplasm or the extracellular fluid and but also for transduction of therapeutic proteins by enhancing membrane-permeability of the recombinant protein using a strong secretional enhancer.

### TECHNICAL FIELD

The present invention relates to a production method for the soluble native form of a recombinant protein by a directional signal (a part of the signal sequence), a secretional enhancer and a protease recognition site.

### BACKGROUND ART

One of the most important applications of modern biotechnology is the production of a recombinant protein, in particular the soluble native form of a recombinant protein. Soluble proteins play an important role in production and recovery of an active form of protein, crystallization for functional studies and industrialization thereof. Recombinant proteins have been expressed in E. coli since E. coli can be easily manipulated, has a rapid growth rate, guarantees stable expression, is economical and easily lends itself to scale-up.

However, when E. coli is used to express a heterologous recombinant protein, the absence of appropriate post-translational chaperones or post-translational processing may cause the expressed protein to misfold and aggregate to form inclusion bodies (Baneyx, Curr. Opin. Biotechnol. 10:411-421, 1999).

Studies have been confirmed that the signal sequence of E. coli directs a foreign polypeptide to the E. coli periplasm (Inouye and Halegoua, CRC Crit. Rev. Biochem. 7:339-371, 1980) and the amino terminal basic region (Lehnhardt et al., J. Biol. Chem. 263:10300-10303, 1988), the hydrophobic region (Goldstein et al., J. Bacteriol. 172:1225-1231, 1990) and the cleavage region (Duffaud and Inouye, J. Biol. Chem. 263:10224-10228, 1988) are all involved in the structure and function of the signal peptide. Several vectors containing signal sequences from E. coli have been developed to produce a soluble protein (ompA: Ghrayeb et al., EMBO J. 3:2437-2442, 1984; Duffaud et al., Methods Enzymol. 153: 492-507, 1987; Delrue et al., Nucleic Acids Res. 16:8726, 1988; phoA: Dodt et al., FEBS Lett. 202:373-377, 1986; Kohl et al., Nucleic Acids Res. 18:1069, 1990; eltA: Morika-Fujimoto et al., J. Biol. Chem. 266:1728-1732, 1991; bla: Oka et al., Agric Biol. Chem. 51:1099-1104, 1987; eltIIb-B: Jobling et al., Plasmid 38:158-173, 1997).

However, all of the signal sequences thus far available on expression vector have only a limited ability to direct soluble protein expression and the use of these vectors results in the production of recombinant fusion proteins having the cleavage region of a signal peptidase, indicating that it is very difficult to produce the native form of a recombinant.

The reason why the production of a recombinant protein using a signal sequence is difficult is that 1) the prediction of the production of a protein in soluble form is impossible, so that many researchers have hypothesized that expression of recombinant proteins in soluble form is inherently dependent on the physical properties of the amino acid sequence; and 2) there are too many sequences acting as a signal sequence but no direct analyzing methods for the function of such signal sequences have been developed (Triplett et al., J. Biol. Chem. 276:19648-19655, 2001).

Thus, the present inventors studied secretional enhancers capable of improving protein secretional efficiency and further completed this invention by confirming that a peptide comprising hydrophilic amino acids linked to a signal sequence containing a basic N-region alone or a basic N-region and central characteristic hydrophobic region can be a secretional enhancer.

### DISCLOSURE

### Technical Problem

It is an object of the present invention to provide a method for producing a soluble recombinant fusion protein effectively from a heterologous gene and a method for recovering the native form of the protein.

### Technical Solution

To achieve the above object, the present invention provides an expression vector containing a gene construct composed of polynucleotide encoding a modified signal sequence consisting of a polypeptide fragment containing an N-region of the signal sequence or a hydrophobic fragment containing the N-region and central characteristic hydrophobic region of the signal sequence and/or a hydrophilic enhancing sequence linked to the N-region fragment and/or the hydrophobic fragment of the signal sequence as a secretional enhancer.

The present invention also provides a recombinant expression vector for the production of a fusion protein containing the modified signal sequence and a heterologous gene.

The present invention further provides a transformant prepared by transforming a host cell with the above expression vector or the recombinant expression vector.

The present invention also provides a method for improving the secretional efficiency of a recombinant protein by using the above transformant.

The present invention also provides a method for producing a recombinant fusion protein.

The present invention also provides a recombinant fusion protein produced by the method of the above.

The present invention also provides a method for producing a heterologous protein.

The present invention also provides a pharmaceutical use of the recombinant fusion protein.

The descriptions of the terms used in the present invention are provided hereinafter.

“Heterologous protein” or “target heterologous protein” indicates the protein that is targeted to be mass-produced by those in the art, precisely every protein that is able to be expressed in a transformant by a recombinant expression vector containing a polynucleotide encoding the target protein.

“Fusion protein” indicates the protein with the addition of another protein or another amino acid sequence in the N-terminal or the C-terminal of the native heterologous protein.

“Signal sequence” indicates the sequence that is involved in efficient directing of a heterologous protein expressed in a virus, a prokaryotic cell or a eukaryotic cell to the periplasm or outside of cells by helping the protein to pass through the cytoplasmic membrane. The signal sequence is composed of the positively charged N-region, the central characteristic hydrophobic region and the C-region with a cleavage site. A signal sequence fragment used in the present invention indicates a part of either one of up to the positively charged N-region, up to the central characteristic hydrophobic region and up to the C-region with a cleavage site or a whole signal sequence.

“Polypeptide” herein indicates the multimer molecule in which at least two amino acids are linked by peptide bond and a protein is also considered as one of the polypeptide.

“Polypeptide fragment” indicates the polypeptide sequence which is in a minimum length or longer with keeping the polypeptide function. If not mentioned otherwise, the polypeptide fragment herein does not include a full-length polypeptide. For example, ‘the polypeptide fragment containing an N-region of the signal sequence’ of the invention indicates a shortened signal sequence functioning as a signal sequence but not a whole signal sequence.

“Polynucleotide” indicates the multimer molecule in which at least two nucleic acids are linked by phosphodiester bond and both DNA and RNA are included.

“Secretional enhancer” indicates the hydrophilic polypeptide composed of hydrophilic amino acids increasing hydrophilicity of the signal sequence.

“N-region” indicates the strong base sequence located at the N-terminal which is well-preserved in general signal sequences and composed of 3-10 amino acids, depending on a signal sequence.

“Central specific hydrophobic region” indicates the region next to an N-region in the general signal sequence structure which is highly hydrophobic by comprising multiple hydrophobic amino acids.

“Modified signal sequence” indicates not a whole signal sequence but the N-region thereof or the polypeptide in which a secretional enhancer is linked to an N-region or a truncated hydrophobic signal peptide comprising an N-region and central specific hydrophobic region or the polypeptide with the addition of a recognition site of a protease in addition to the above.

“Signal sequence fragment” or “truncated signal sequence” indicates the part of a signal sequence. If not mentioned otherwise herein, this fragment indicates the fragment excluding the C-terminal region from the signal sequence.

“Restriction enzyme site” indicates the polynucleotide sequence recognized and digested by a DNA restriction enzyme, if not mentioned otherwise.

“Recognition site of protease” indicates the amino acid sequence recognized and digested by a protease.

“Amphipathic domain” indicates the domain having both the hydrophobic and hydrophilic regions, which is the region having a transmembrane domain-like structure. So, in the present invention, the amphipathic domain is understood as a “transmembrane-like domain”.

“Transmembrane-like domain” indicates a predicted region from the amino acid sequence that is expected to have a similar structure to the transmembrane domain of membrane protein (Brasseur et al., Biochim. Biophys. Acta 1029(2): 267-273, 1990). In general, the transmembrane-like domain is easily predicted by various computer soft wares predicting a transmembrane domain. And the soft-wares are exemplified by TMpred (//www.ch.embnet.org/software/TMPRED_form.html), HMMTOP (//www.enzim.hu/hmmtop/html/submit.html), TBBpred (//www.imtech.res.in/raghava/tbbpred/), DAS-TMfilter (://www.enzim.hu/DAS/DAS.html), etc. The “transmembrane-like domain” includes a transmembrane domain identified to have an actual membrane potential.

“Expression vector” indicates the linear or circular DNA molecule comprising a fragment encoding a target polypeptide operably linked to an additional fragment provided for transcription of the expression vector. The additional fragment includes a promoter and a termination codon. The expression vector includes one or more replication origins, one or more selection markers, an enhancer, a polyadenylation signal, etc. The expression vector is generally derived from a plasmid or a virus DNA or both.

“Operably linked” indicates that fragments are arranged and linked to operate as intended, for example transcription is started at a promoter and terminated at a termination codon.

“Promoter” indicates the gene part to which RNA polymerases bind to start mRNA synthesis.

“Host cell” indicates the cell that is infected by a gene carrier such as a virus or a plasmid vector in order to produce a recombinant protein or a heterologous protein.

“Blood-brain barrier” indicates the functional barrier to interrupt the invasion of a specific material into brain from blood. The main structure of the blood-brain barrier is presumed to be a tight junction (zonula occludens) in capillary endothelial cells.

Hereinafter, the present invention is described in detail.

The present inventors first constructed a vector to express a fusion protein in soluble form to produce an adhesive protein Mefp1 (Waite et al., Biochemistry 24:5010-5014, 1985) using a signal sequence, precisely by connecting a heterologous gene of mefp1 and the coding sequence of the whole and a part of OmpA signal peptide (OmpASP) as a signal sequence by PCR, based on His tagged pET vector, and then constructed a vector to obtain a native N-terminal form of Mefp1 protein in soluble form by ligating a heterologous gene to the modified signal sequence with OmpASPₜᵣ-factor Xa cleabage in which the truncated OmpASP (OmpASPₜᵣ) and the factor Xa recognition site are linked. And at last, the inventors produced the native form of Mefp1 protein after treating with factor Xa protease to cleave off the modified signal sequence. The present inventors further confirmed that the whole or/and a part of OmpASP has a regular pI value and this pI value is very important in expression of a soluble protein.

In the expression experiment, olive flounder Hepcidin I was failed to be expressed as a soluble fusion protein with OmpASPₜᵣ. So, in the case that a heterologous protein was not expressed in soluble form by a signal sequence, the sequences encoding such amino acids as Arg and Lys having high pI and hydrophilicity were inserted as a secretional enhancer into the C-terminal region of a signal sequence, leading to the fusion of the coding sequence of a recognition site of protease with a heterologous gene by PCR. After constructing a vector as the above, the inventors produced a soluble protein. At this time, the upstream of the heterologous gene was referred as ‘modified signal sequence region’.

The modified signal sequence was designed in the form of OmpASPₜᵣ-SmaI-Xa (in the case of Mefp1) or OmpASPₜᵣ-( )-Xa (in the case of olive flounder (Paralichthys olivaceus) Hepcidin I) and six different amino acids associated with the characteristics of pI and hydrophobicity/hydrophilicity were selected and inserted in SmaI or -( )- region by six homologous amino acid sequence of six per each amino acid, resulting in the construction of clones. Then, the expression was investigated. As a result, the expression of a soluble protein was increased in the clone with the insertion of the sequence corresponding to Arg and Lys having high pI value and hydrophilicity. The expression of a soluble protein was slightly increased in the case of a soluble Mefp1, while the expression was significantly increased in the case of a soluble olive flounder Hepcidin I, indicating the inserted amino acids Arg and Lys acted as a secretional enhancer. In conclusion, the insertion of Arg and Lys, basic amino acids, in the C-terminal increases pI value and hydrophilicity of a signal sequence and thereby increases the expression of a soluble protein.

It was also confirmed that the shorter the N-terminal sequence of a signal sequence against the amount of Arg and Lys having a high pI value and hydrophilicity in the C-terminal, the higher the hydrophilicity of the signal sequence and the more the expression of a soluble target protein were observed. So, high pI value and hydrophilicity in the modified signal sequence region are the key factors for the expression of a soluble protein and hydropathy profile might be a secondary key. If a signal sequence is designed to be longer than a certain length, this sequence will have a transmembrane-like domain structure having a higher hydrophilicity than that of a general transmembrane domain or transmembrane-like domain, and this structure enables the expression of a soluble protein.

Hydropathy profiles of the signal sequence regions of the soluble clones are investigated. As a result, the signal sequence of such clone has a transmembrane-like domain having a similar or higher hydrophilic profile than the amphipathic domain or transmembrane-like domain in olive flounder Hepcidin I. This result indicates that a signal sequence requires a transmembrane-like domain having a higher hydrophilicity in order to express a heterologous protein containing amphipathic domain such as the molecule of olive flounder Hepcidin I.

Therefore, hydrophobicity/hydrophilicity average value of a signal sequence has been proved to be a critical factor for the expression of a soluble protein. The hydrophobicity/hydrophilicity average value (Hopp & Woods scale) of the modified signal sequence can be predicted and the hydropathy profile can be optimized by the computer program DNASIS™ (Hitachi, Japan, 1997), so that a sequence having a transmembrane-like domain having a higher hydrophilicity than a target heterologous protein can be designed to express a soluble protein.

The present invention is described in more detail hereinafter.

The present inventors constructed pET-22b(+)[ompASP_{( )}-7×mefp1*] clone by PCR using the template presented in FIG. 2 by the fusion of the 5′-end of 7×mefp1 encoding a heterologous protein with the coding sequence of a region from OmpASP₁₋₃, the part of a signal sequence OmpA inducing secretion in E. coli, to the whole coding sequence of OmpASP₁₋₂₃ (see Table 1). The constructed vector clone was transformed into E. coli BL21(DE3) and the expression of a target protein was induced for 3 hours using IPTG. As a result, the clones constructed above all expressed soluble recombinant MefpI in E. coli (see Table 1 and FIG. 3)

A signal sequence has the arrangement of a positively charged N-region starting from Met, a central characteristic hydrophobic region and a C-region ending with a cleavage site. The signal sequence regulates folding of a precursor protein and plays a key role in protein secretion (Izard et al., Biochemistry 34:9904-9912, 1995; Wickner et al., Annu. Rev. Biochem. 60:101-124, 1991).

As of today, pI value, hydrophobicity, molecular weight and stability of a whole protein have been known as critical factors affecting the expression of a recombinant protein in soluble form. The present inventors prepared modified signal sequences and investigated pI values from the whole and a part of a signal sequence OmpASP, which is from OmpASP₁₋₃, to the whole OmpASP₁₋₂₃. As a result, pI values of them were all 10.55, regardless of the lengths of them (Table 2). All clones were treated with IPTG for 3 hours to induce the expression of a soluble target protein and the result showed that they all produced soluble Mefp1, regardless of the length of OmpASP (see FIG. 3). The above result indicates that not hydrophobicity but high pI value acts as a directional signal for the expression of soluble Mefp1 not only in a part of OmpASP but also in the whole OmpASP. This result also indicates that the positively charged N-region alone can express nascent polypeptide chains in soluble form, which was the astonishing founding first made by the present inventors. The N-region of a signal sequence happens to contain glutamic acid or aspartic acid instead of a positively charged basic amino acid, and in this case, pI value might be up to 4. Even so, the N-region can be used as a directional signal sequence. The preferable pI value of the modified signal sequence is at least 8 and more preferably at least 9 and most preferably at least 10.

In the present invention, E. coli originated OmpA signal sequence was used, but signal sequences having a OmpA signal sequence-like structure such as CT-B (cholera toxin subunit B) signal sequence, LTπb-B (E. coli heat-labile enterotoxin B subunit) signal sequence, BAP (bacterial alkaline phosphatase) signal sequence (Izard and Kendall, Mol. Microbiol. 13:765-773, 1994), Yeast carboxypeptidase Y signal sequence (Blachly-Dyson and Stevens, J. Cell. Biol. 104:1183-1191, 1987), Kluyveromyces lactis killer toxin gamma subunit signal sequence (Stark and Boyd., EMBO J. 5(8): 1995-2002, 1986), bovine growth hormone signal sequence (Lewin, B. (Ed), GENES V, p 290. Oxford University Press, 1994), influenza neuraminidase signal-anchor (Lewin, B. (Ed), GENES V, p 297. Oxford University Press, 1994), Translocon-associated protein subunit alpha (TPAP-α) (Prehn et al., Eur. J. Biochem. 188(2): 439-445, 1990) signal sequence and Twin-arginine translocation (Tat) signal sequence (Robisnon, Biol. Chem. 381(2): 89-93, 2000) can also be used. In addition, any other virus, prokaryote and eukaryotic signal sequences and leader sequences having a similar structure to that of the above can be used. All of the above sequences have high hydrophobicity.

To produce a recombinant fusion protein, the C-terminal of the modified signal sequence region having a protease recognition site provides a site for the fusion of a heterologous protein. Once a recombinant protein is expressed, it is treated with a protease, leading to the recovery of a native form of the heterologous protein. Based on the above results, the present inventors designed to fuse the recognition site of factor Xa protease, for cutting the C-terminal end of the recognition, to OmpASP₁₋₈ and constructed pET-22b(+) (ompASP₁₋₈-Xa-7×mefp1*) clone by PCR using 7×mefp1 as a template (FIG. 2) and the expression of the clone in E. coli was investigated (Table 1). As a result, the clone produced a soluble protein. It was further confirmed that the modified signal sequence used as a directional signal sequence was eliminated by treating with the protease factor Xa and the native form of MefpI was obtained (see FIG. 4).

The recognition site of factor Xa protease used in the present invention has preferably the sequence of Ile-Glu-Gly-Arg.

The recognition site of protease of the invention is preferably selected from a group consisting of factor Xa protease, enterokinase (Asp-Asp-Asp-Asp-Lys) genenase I (His-Tyr) and furin (Arg-X-X-Arg).

The present inventors investigated the functions of the native form of protein recovered form the expressed recombinant. Adhesive property of the recombinant Mefp1 was tested. As a result, the recombinant Mefp1 had excellent adhesive property, compared with the control BSA (see FIG. 5). Therefore, the production method of a recombinant protein of the present invention was confirmed to be effective in production of a heterologous protein in soluble native form without damaging the functions thereof.

To investigate the effect of the modified signal sequence in any other regions than OmpASP fragment on soluble Mefp1 expression, the present inventors selected a SmaI site for cloning blunt-end DNA fragments conveniently, designed the signal sequence as OmpASP₁₋₈-SmaI-Xa, and constructed pET-22b(+)(ompASP₁₋₈-SmaI-Xa-7×mefp1*) clone with PCR (see Table 1). A clone with the insertion of an amino acid having a high pI and hydrophilicity such as Arg or Lys in the SmaI site was also constructed. The clone containing the amino acid having a high pI and hydrophilicity was also confirmed to express a recombinant Mefp1 and in fact the secretion thereof was somewhat increased.

In another experimental embodiment, olive flounder Hepcidin I was not expressed as a soluble fusion protein by OmpASPₜᵣ (see Table 3).

To screen a secretional enhancer, the present inventors designed the signal sequence region as OmpASP₁₋₁₀-( )-Xa and inserted up to 6 homologous sequences of the selected amino acids affecting pI value and hydrophobicity/hydrophilicity, which are 6×Arg, 6×Lys, 6×Glu, 6×Asp, 6×Tyr, 6×Phe, 6×Trp, into the ( ) site (see Table 4). PCR was performed using olive flounder Hepcidin I gene (Kim et al., Biosci. Biotechnol. Biochem. 69:1411-1414, 2005) as a template to construct pET-22b(+)[ompASP₁₋₁₀-( )-Xa-ofhepcidinI**] clone (see Table 3). The clones were tested in E. coli. Those lones having 6×Arg and 6×Lys with high pI and hydrophilicity expressed soluble olive flounder Hepcidin I very strongly, while other clones inserted with other amino acids expressed soluble olive flounder Hepcidin I very weakly (see FIG. 6). The above results suggest that the expression of soluble olive flounder Hepcidin I is associated with high pI values and hydrophilic amino acids Arg and Lys, and therefore proved that Arg and Lys inserted into the C-terminal of a signal sequence acted as a secretional enhancer (see Table 4).

The present inventors further investigated the effect of the modified signal sequence region with the various length of OmpASP fragment in the N-terminal and the various form of -( )-Xa in the C-terminal on hydrophilicity. First, the N-terminal signal sequence OmpASP is prepared in various lengths, which were attached to the C-terminal —6×Arg-Xa, followed by PCR to construct pET-22b(+)[ompASP(−6×Arg-Xa-ofhepcidinI**] (see Table 3). The clones were tested in E. coli. As a result, as the length of the OmpASP sequence decreased, hydrophilicity was increased by the Hopp & Woods scale (Example 6) and the yield of the soluble target protein was increased (see FIG. 7). The Hopp & Woods scale hydropathy profile also revealed that the OmpASP₁₋₆-6×Arg-Xa attached with the shortest N-region sequence of OmpASP₁₋₆ exhibited only a hydrophilic curve. When the signal sequence longer than OmpASP₁₋₈ attached to the −6×Arg-Xa, the resultant signal sequence exhibited a hydrophobic curve in the N-terminal and a hydrophilic curve in the C-terminal, which was resemble with the general transmembrane-like domain. From the above results it was confirmed that the addition of an amino acid with a strong hydrophilicity to the C-terminal of a hydrophobic fragment composed of a basic N-region and central characteristic hydrophobic region results in a transmembrane-like domain structure and when the hydrophilicity in the C-terminal of the signal sequence region is larger than that of transmembrane domain or transmembrane-like domain or amphipathic domain of nascent target polypeptide chains, the nascent target polypeptide chains are able to be expressed in soluble form. This founding was first made by the present inventors, which is astonishing result. Based on the method of the invention, those proteins generally not expressed in soluble form such as membrane proteins can now be expressed in soluble form, which can further contribute to improvement of membrane permeability of various proteins applicable as a biological agent with the increase of drug delivery. In relation to drug delivery, the conventional protein drugs have a common disadvantage of not passing through blood-brain barrier. But, according to the method of the invention, this disadvantage can be overcome, indicating the realization of effective drug delivery. That is, a therapeutic protein (for example, anti-beta-amyloid antibody) for various brain diseases can be directly injected through the blood vessel instead of injecting directly into the cerebral ventricle.

The present inventors set the length of a signal sequence as OmpASP₁₋₁₀ in the N-terminal, attached 2˜10 hydrophilic amino acids to the C-terminal of the -( )-Xa region, and followed by PCR to construct the general clone of pET-22b(+)[ompASP₁₋₁₀-( )-Xa-ofhepcidinI**] (see Table 3). The constructed clones were expressed in E. coli. As the amount of hydrophilic amino acids attached to the C-terminal of the signal sequence region (the modified signal sequence), the Hopp & Woods scale hydrophilicity was increased (Example 6), which was paralleled with the increased yield of a soluble target protein (see FIG. 8). According to the Hopp & Woods scale hydropathy profile, every signal sequence expressing a soluble form of a protein exhibited a hydrophobic curve in the N-terminal region and a hydrophilic curve in the C-terminal region, indicating a transmembrane-like domain structure was formed.

So, the modified signal sequence increases hydrophilicity and thereby enables the expression of a target protein in soluble form in the above two cases, suggesting that the Hopp & Woods scale hydrophilicity might be used as indexes for soluble expression of a target protein. pI value of OmpASP fragment originated from the N-region of a signal sequence is closely involved in a directional signal and hydrophilicity level of the -( )-Xa in the C-terminal is important to determine the role of a secretional enhancer. If the length of the N-terminal region is set as OmpASP₁₋₁₀ and the C-terminal region is modified, every signal sequence expressing a soluble protein will exhibit a hydrophobic curve in the N-terminal region and a hydrophilic curve in the C-terminal region, which is a transmembrane domain-like hyperbolic curve. So, the hydropathy profile according to the Hopp & Woods scale can be used as a secondary index.

The hydropathy profile of olive flounder Hepcidin I (without ** region) and a signal sequence by Hopp & Woods scale thereof were simulated by using a computer program (see FIG. 9). The control olive flounder Hepcidin I molecule had an amphipathic domain (FIG. 9A), while the hypothetical signal sequence-olive flounder Hepcidin I fusion protein included two transmembrane-like domains; one in the signal sequence and the other in olive flounder Hepcidin I region (FIGS. 9B, 9C and 9D). The recombinant olive flounder Hepcidin I expressed strongly in soluble form contained a transmembrane-like domain having a higher hydrophilicity in the signal sequence than the amphipathic domain of Hepcidin I (FIG. 9D). The clone pET-22b(+)[ompASP₁₋₁₀-6×Arg-Xa-ofhepcidinI**] corresponding to the fusion protein of FIG. 9D was expressed in soluble form (see FIG. 8 lane 4). Therefore, it was confirmed that a signal sequence having a transmembrane-like domain with a higher hydrophilicity than the general transmembrane-like domain of the target molecules is required to express such molecules having one or more of transmembrane domain, transmembrane-like domain or amphipathic domain in soluble form to overcome the barrier. To predict the expression of a soluble target protein, the Hopp & Woods scale hydrophobicity/hydrophilicity and hydropathy profiles can be used as indexes.

Therefore, the method of the present invention can be effectively used for the production of a soluble heterologous protein with a native N-terminal form.

### DESCRIPTION OF DRAWINGS

The application of the preferred embodiments of the present invention is best understood with reference to the accompanying drawings, wherein:

FIG. 1 is a schematic diagram illustrating various exemplary embodiments on the expression vector of the invention.

FIG. 2 is a diagram illustrating the sequence of the cloned mefp1 clone, pBluescriptIISK(+)-La-7×mefp1-Ra:

La (left-adaptor): underlined BamHI/EcoRI/SmaI region;

Linker: linker DNA (TACAAA);

AlaLysProSerTyrProProThrTyrLys: a basic unit of Mefp1; and

Ra (right adaptor): underlined Arg/HindIII/SalI/XhoI region.

FIG. 3 is a diagram illustrating the expression of the recombinant Mefp1 fusion protein, induced from pET-22b(+)[ompASP_{( )}-7×mefp1*] (*: Ra-6×His) clone, in soluble supernatant, and anti-His tag antiserum was used to detect the recombinant Mefp1 produced by pET-22b(+) containing the coding sequence of His tag in the 3′-end:

(A) SDS-PAGE;

(B) Western blotting;

Right upper arrow: recombinant Mefp1;

Right lower arrow: Mefp1 with OmpA signal sequence (OmpASP) cleavage (matured form with OmpASP₁₋₂₁ cleavage by OmpA signal peptidase);

Lane 1: OmpASP₁₋₃-7×Mefp1*;

Lane 2: OmpASP₁₋₅-7×Mefp1*;

Lane 3: OmpASP₁₋₇-7×Mefp1*;

Lane 4: OmpASP₁₋₉-7×Mefp1*;

Lane 5: OmpASP₁₋₁₁-7×Mefp1*;

Lane 6: OmpASP₁₋₁₃-7×Mefp1*;

Lane 7: OmpASP₁₋₁₅-7×Mefp1*;

Lane 8: OmpASP₁₋₂₁-7×Mefp1* (half of OmpASP₁₂₁ was cleaved by OmpA signal peptidase but the other half was not since OmpA signal sequence was attached to Mefp1 sequence as some of the sequence was absent); and

Lane 9: OmpASP₁₋₂₃-7×Mefp1* (OmpASP₁₂₁ was completely cleaved by OmpA signal peptidase because OmpA signal sequence was fully preserved).

FIG. 4 is a diagram illustrating the expression of the soluble recombinant Mefp1 protein produced from the clone pET-22b(+) (ompASP₁₋₈-Xa-7×mefp1*) (*: Ra-6×His) and 7×Mefp1* with a native form of amino acid terminus:

(A) SDS-PAGE;

(B) Western blotting;

Right upper arrow: recombinant Mefp1 (OmpASP₁₋₈-Xa-7×Mefp1*);

Right lower arrow: native form Mefp1 (7×Mefp1*);

Lane 1: non-induced whole cells for 3 h;

Lane 2: expression-induced whole cells for 3 h;

Lane 3: expression-induced soluble supernatant fraction for 3 h; and

Lane 4: Mefp1 with a native N-terminal region produced by treating the three-hour expression-induced soluble supernatant fraction with factor Xa protease.

FIG. 5 is a diagram illustrating the coating of the recombinant protein Mefp1 on a glass slide. +: treatment of proteins with tyrosinase; and

−: treatment of proteins without tyrosinase.

FIG. 6 illustrates a secretional enhancer of OmpASPₜᵣ-( )-Xa for the expression of the recombinant olive flounder (Paralichthys olivaceus) Hepcidin I (ofHepcidinI) from pET22b(+)[ompASP₁₋₁₀-( )-Xa-ofhepcidinI**] Glu/HindIII/Sal I/Xho I-6×His) clone. As shown in Table 4, pI value and hydrophobicity/hydrophilicity value are associated with the amino acids inserted in the parenthesis of OmpASP₁₋₁₀-( )-Xa:

(A) SDS-PAGE;

(B) Western blotting;

Arrow: recombinant ofHepcidin I;

M: marker;

Lane 1: control;

Lane 2: 6×Arg;

Lane 3: 6×Lys;

Lane 4: 6×Glu;

Lane 5: 6×Asp;

Lane 6: 6×Tyr; and

Lane 7: 6×Trp.

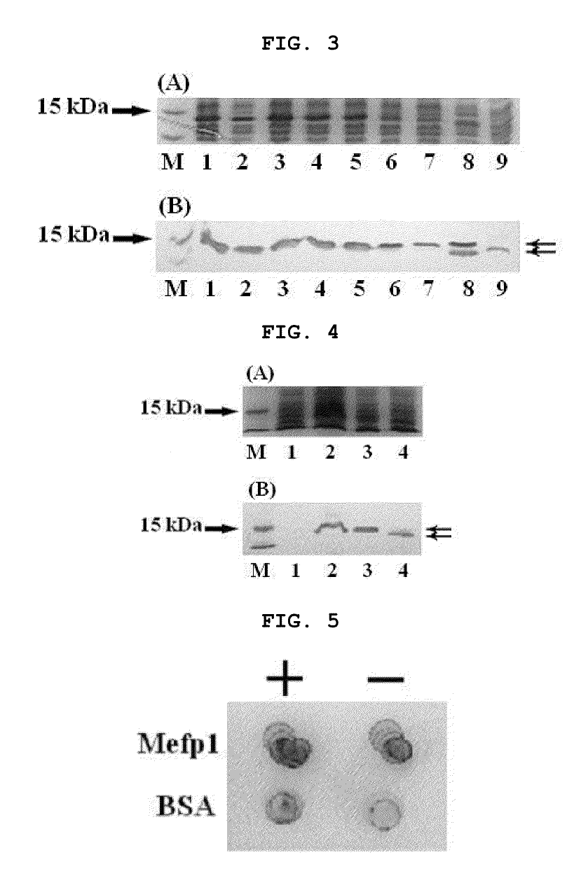

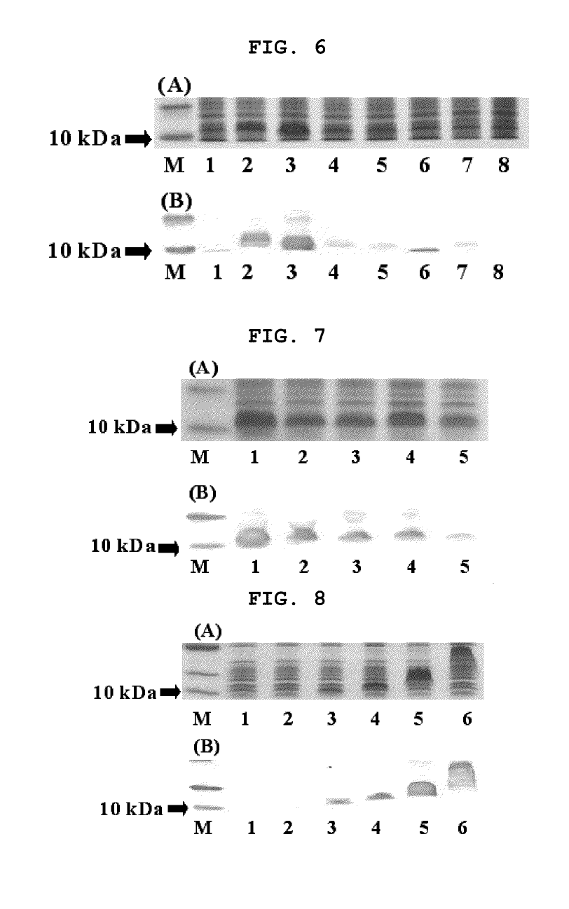

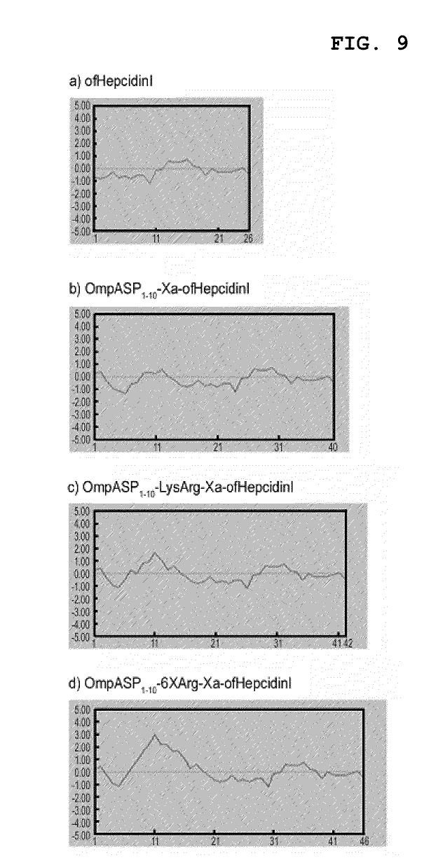

FIG. 7 is a diagram illustrating the effect of the length of OmpASP, as a directional signal, on the expression ofHepcidin I in soluble form. The soluble supernatant fraction was induced with IPTG for 3 hours. Western blotting was performed as described in FIG. 3:

(A) SDS-PAGE;

(B) Western blotting;

Arrow: recombinant ofHepcidin I;

M: marker;

Lane 1: pET22b(+)[ompASP₍₁₋₆₎-6×Arg-Xa-ofhepcidinI**];

Lane 2: pET22b(+)[ompASP₍₁₋₈₎-6×Arg-xa-ofhepcidinI**];

Lane 3: pET22b(+)[ompASP₍₁₋₁₀₎-6×Arg-Xa-ofhepcidinI**];

Lane 4: pET22b(+)[ompASP₍₁₋₁₂₎-6×Arg-Xa-ofhepcidinI**]; and

Lane 5: pET22b(+)[ompASP₍₁₋₁₄₎-6×Arg-Xa-ofhepcicdinI**].

FIG. 8 is a diagram illustrating the effect of high pI value and hydrophilic amino acids in a signal sequence on the expression ofHepcidin I. The soluble supernatant fraction was induced with IPTG for 3 hours. Western blotting was performed as described in FIG. 3:

(A) SDS-PAGE;

(B) Western blotting;

Arrow: recombinant ofHepcidin I;

M: marker;

Lane 1: control; pET22b(+)[ompASP₁₋₁₀-Xa-ofhepcidinI**];

Lane 2: pET22b(+)[ompASP₁₋₁₀-(LysArg)-Xa-ofhepcidinI**];

Lane 3: pET22b(+)[ompASP₁₋₁₀-(4×Arg)-Xa-ofhepcidinI**];

Lane 4: pET22b(+)[ompASP₁₋₁₀-(6×Arg)-Xa-ofhepcidinI**];

Lane 5: pET22b(+)[ompASP₁₋₁₀-(8×Arg)-Xa-ofhepcidinI**]; and

Lane 6: pET22b(+)[ompASP₁₋₁₀-(10×Arg)-Xa-ofhepcidinI**].

FIG. 9 illustrates the simulated hydropathy profile by the Hopp & Woods scale using a computer program in ofHepcidin I and its variants containing the hydrophilic amino acids in OmpASP₁₋₁₀-( )-Xa:

(A) ofHepcidin I (26 aa, Av −0.21);

(B) OmpASP₁₋₁₀-Xa-ofHepcidinI (40 aa, Av −0.19);

(C) OmpASP₁₋₁₀-LysArg-Xa-ofHepcidinI (42 aa, Av −0.04);

(D) OmpASP₁₋₁₀-6×Arg-Xa-ofHepcidinI (46 aa, Av 0.22);

aa: amino acid number; and

Av: hydrophobicity/hydrophilicity average value.

### MODE FOR INVENTION

Hereinafter, the preferable embodiments of the invention are described in detail.

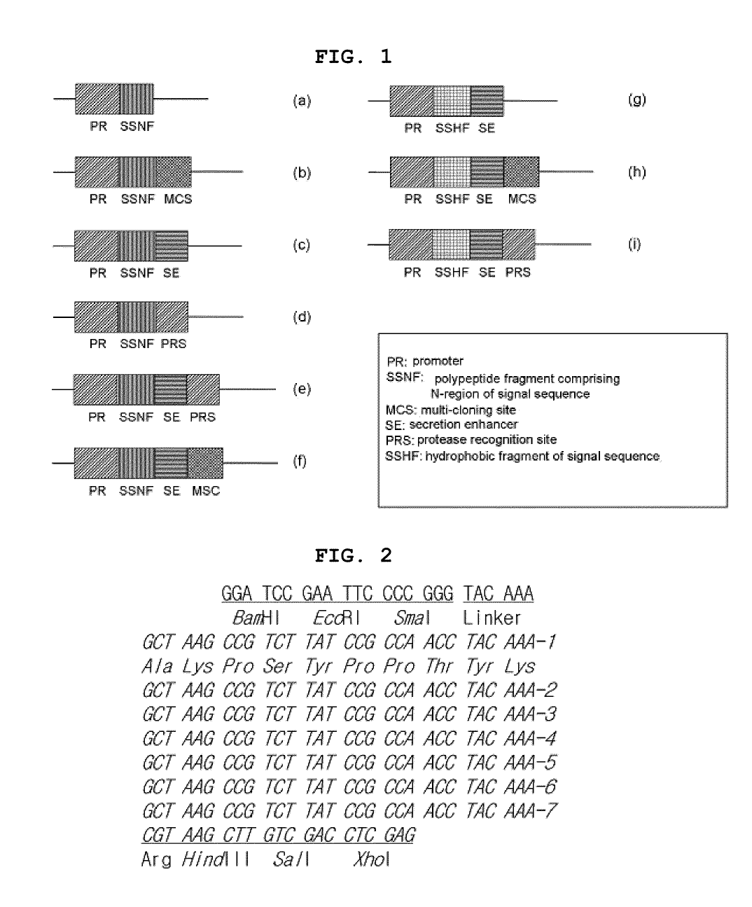

The present invention provides an expression vector for increasing secretional efficiency of a heterologous protein containing a gene construct composed of (i) a promoter, and (ii) a polynucleotide encoding the N-region of a signal sequence operably linked to the promoter (see FIG. 1(a)).

Herein, the promoter is preferably a viral promoter, a prokaryotic promoter or a eukaryotic promoter. The viral promoter is preferably one of cytomegalovirus (CMV) promoter, polyomavirus promoter, fowl pox virus promoter, adenovirus promoter, bovine papillomavirus promoter, rous sarcomavirus promoter, retrovirus promoter, hepatitis B virus promoter, herpes simplex virus thymidine kinase promoter and simian virus 40 (SV40) promoter, but not always limited thereto. The prokaryotic promoter is preferably one of T7 promoter, SP6 promoter, heat-shock protein 70 promoter, β-lactamase, lactose promoter, alkaline phosphatase promoter, tryptophane promoter and tac promoter, but not always limited thereto. The eukaryotic promoter is preferably a yeast promoter, a plant promoter or an animal promoter. The yeast promoter herein is preferably selected from a group consisting of 3-phosphoglycerate kinase promoter, enolase promoter, glyceraldehyde-3-phosphate dehydrogenase promoter, hexokinase promoter, pyruvate dicarboxylase promoter, phosphofructokinase promoter, glucose-6-phosphate isomerase promoter, 3-phosphoglycerate mutase promoter, pyruvate kinase promoter, triosphosphate isomerase promoter, phosphoglucose isomerase promoter, glucokinase promoter, alcohol dehydrogenase 2 promoter, isocytochrome C promoter, acidic phosphatase promoter, Saccharomyces cerevisiae GALL promoter, Saccharomyces cerevisiae GAL7 promoter, Saccharomyces cerevisiae GAL10 promoter and Pichia pastoris AOX1 promoter, but not always limited thereto. The animal promoter is preferably selected from a group consisting of a heat-shock protein promoter, a proactin promoter and an immunoglobulin promoter, but not always limited thereto. In the present invention, the promoter can be any promoter that is able to express a foreign gene normally in a host cell.

The signal sequence herein is preferably a viral, a prokaryotic or a eukaryotic signal sequences or leader sequences, which are exemplified by OmpA signal sequence, CT-B (cholera toxin subunit B) signal sequence, LTπb-B (E. coli heat-labile enterotoxin B subunit) signal sequence, BAP (bacterial alkaline phosphatase) signal sequence (Izard and Kendall, Mol. Microbiol. 13:765-773, 1994), yeast carboxypeptidase Y signal sequence (Blachly-Dyson and Stevens, J. Cell. Biol. 104:1183-1191, 1987), Kluyveromyces lactis killer toxin gamma subunit signal sequence (Stark and Boyd. EMBO J. 5(8): 1995-2002, 1986), bovine growth hormone signal sequence (Lewin, B. (Ed), GENES V, p 290. Oxford University Press, 1994), influenza neuraminidase signal-anchor (Lewin, B. (Ed), GENES V, p 297. Oxford University Press, 1994), translocon-associated protein subunit alpha (TRAP-α) (Prehn et al., Eur. J. Biochem. 188(2): 439-445, 1990) signal sequence and Twin-arginine translocation (Tat) signal sequence (Robisnon, Biol. Chem. 381(2): 89-93. 2000), but not always limited thereto and any signal sequence harboring a high basic N-region can be included.

The polypeptide fragment containing the N-region is preferably composed of peptides with different lengths from 3 to 21 amino acids necessarily including the 1^{st}-3^{rd} amino acids of a signal sequence, and the length of the fragment can be determined by considering pI value and hydropathy profile of the N-region of the signal sequence of the invention. According to a preferred embodiment of the present invention, pI value of the polypeptide fragment containing the signal sequence N-region is at least 8 and more preferably at least 9 and most preferably at least 10. The N-region contains at least two basic amino acids selected among positively charged amino acids such as lysine or arginine and negatively charged amino acids such as aspartic acid or glutamic acid and pI value with the positively charged amino acids is preferably at least 8 and pI value with negatively charged amino acids is up to 4. Every signal sequence exhibiting the N-region pI value of at least 8 can be used as a polypeptide fragment for an expression vector, but not always limited thereto.

One or more amino acids of the N-region of a signal sequence can be substituted with other basic amino acids such as arginine and lysine. If one or more amino acids having high pI values such as arginine and lysine reside in the N-region, secretional efficiency will be increased. And this substitution method has been well known to those in the art (Sambrook et al., 1989. “Molecular Cloning: A Laboratory Manual”, 2nd ed. Cold Spring Harbor Laboratory Press, Cold Spring Harbor, N.Y.).

A polynucleotide encoding a secretional enhancer can be operably linked to another polynucleotide encoding the polypeptide fragment containing the N-region of the vector of the invention (see FIG. 1(c)). A secretional enhancer comprises high pI values and hydrophilic amino acids, so it can increase hydrophilicity of a signal sequence to accelerate the direction of a heterologous protein to the periplasm. The secretional enhancer is a hydrophilic peptide composed of at least 60% of hydrophilic amino acids. Thus, it is preferred for a secretional enhancer to contain hydrophilic amino acids at least 60%, more preferably at least 70%, and the length is not limited but generally 2-50 amino acids long and more preferably 4-25 amino acids long and most preferably 6-15 amino acids long. It is most preferred for a secretional enhancer to be composed of 6 hydrophilic amino acid repeat. pI value of a secretional enhancer is not limited but preferably at least 10.

In a preferred embodiment of the present invention, a polynucleotide encoding a protease recognition site was operably linked to another polynucleotide encoding the polypeptide containing the N-region of the expression vector of the invention (see FIG. 1(d)). The protease recognition site herein can be one of factor Xa recognition site, enterokinase recognition site, genenase I recognition site and furin recognition site or two or more recognition sites are linked stepwise. And if factor Xa protease is used, the recognition site, Ile-Glu-Gly-Arg is preferred.

In another preferred embodiment of the present invention, the polynucleotide encoding a secretional enhancer is inserted in between the polynucleotide encoding a polypeptide fragment containing the N-region and the polynucleotide encoding a protease recognition site in an expression vector (see FIG. 1(e)). This insertion is preferably performed using a restriction enzyme site cut by a restriction enzyme generating a blunt end such as SmaI. The protease recognition site is one or more selected from a group consisting of factor Xa recognition site, enterokinase recognition site, genenase I recognition site and furin recognition site.

In another preferred embodiment of the present invention, the expression vector of the present invention additionally includes a restriction enzyme site for the insertion of a gene encoding a heterologous protein (see FIGS. 1(b) and (f)). This restriction enzyme site is inserted next to the polynucleotide encoding the polypeptide fragment containing the N-region of a signal sequence (FIG. 1(b)). If the vector includes a polynucleotide encoding a secretional enhancer, the restriction enzyme site is inserted next to the polynucleotide (FIG. 1(f)). If an expression vector includes a polynucleotide encoding a protease recognition site, a restriction enzyme site might be or not be inserted, and in fact the cloning of a gene encoding a heterologous protein to obtain a native form by taking advantage of a restriction enzyme site is not desirable.

In the meantime, a gene encoding a heterologous protein can be inserted into one or more vectors described above. At this time, the heterologous protein is not limited to a specific protein and any protein regarded as acceptable by those in the art can be used. For example, a protein selected from a group consisting of an antigen, an antibody, a cell receptor, an enzyme, a structural protein, a serum, and a cell protein can be expressed as a recombinant fusion protein. The heterologous protein preferably does not contain a transmembrane domain, transmembrane-like domain or amphipathic domain inside. The protein without a transmembrane domain, transmembrane-like domain or amphipathic domain is not limited but Mefp1 multimer is preferred.

The present invention provides an expression vector for increasing secretional efficiency of a heterologous protein containing a gene construct composed of (i) a promoter, (ii) a polynucleotide encoding a hydrophobic fragment comprising the N-region and central characteristic hydrophobic region of a signal sequence operably linked to the promoter, and (iii) a polynucleotide encoding a secretional enhancer operably linked to the polynucleotide of (ii) (see FIG. 1(g)).

The promoter for the expression vector of the invention is preferably selected from a group consisting of a viral promoter, a prokaryotic promoter, and a eukaryotic promoter, but not always limited thereto. The viral promoter herein is preferably selected from a group consisting of cytomegalovirus (CMV) promoter, polyomavirus promoter, fowl pox virus promoter, adenovirus promoter, bovine papillomavirus promoter, rous sarcomavirus promoter, retrovirus promoter, hepatitis B virus promoter, herpes simplex virus thymidine kinase promoter and simian virus 40 (SV40) promoter, but not always limited thereto. The prokaryotic promoter is preferably selected from a group consisting of T7 promoter, SP6 promoter, heat-shock protein 70 promoter, β-lactamase, lactose promoter, alkaline phosphatase promoter, tryptophane promoter and tac promoter, but not always limited thereto. The eukaryotic promoter is preferably a yeast promoter, a plant promoter or an animal promoter. The yeast promoter herein is preferably selected from a group consisting of 3-phosphoglycerate kinase promoter, enolase promoter, glyceraldehyde-3-phosphate dehydrogenase promoter, hexokinase promoter, pyruvate dicarboxylase promoter, phosphofructokinase promoter, glucose-6-phosphate isomerase promoter, 3-phosphoglycerate mutase promoter, pyruvate kinase promoter, triosphosphate isomerase promoter, phosphoglucose isomerase promoter, glucokinase promoter, alcohol dehydrogenase 2 promoter, isocytochrome C promoter, acidic phosphatase promoter, Saccharomyces cerevisiae GALL promoter, Saccharomyces cerevisiae GAL7 promoter, Saccharomyces cerevisiae GAL10 promoter and Pichia pastoris AOX1 promoter, but not always limited thereto. The animal promoter is preferably selected from a group consisting of a heat-shock protein promoter, a proactin promoter and an immunoglobulin promoter, but not always limited thereto.

The signal sequence included in the expression vector of the invention is preferably a viral, a prokaryotic or a eukaryotic signal sequences or leader sequences, which are exemplified by OmpA signal sequence, CT-B (cholera toxin subunit B) signal sequence, LTπb-B (E. coli heat-labile enterotoxin B subunit) signal sequence, BAP (bacterial alkaline phosphatase) signal sequence (Izard and Kendall, Mol. Microbiol. 13:765-773, 1994), yeast carboxypeptidase Y signal sequence (Blachly-Dyson and Stevens, J. Cell. Biol. 104:1183-1191, 1987), Kluyveromyces lactis killer toxin gamma subunit signal sequence (Stark and Boyd, EMBO J. 5(8): 1995-2002, 1986), bovine growth hormone signal sequence (Lewin, B. (Ed), GENES V, p 290. Oxford University Press, 1994), influenza neuraminidase signal-anchor (Lewin, B. (Ed), GENES V, p 297. Oxford University Press, 1994), translocon-associated protein subunit alpha (TPAP-α) (Prehn et al., Eur. J. Biochem. 188(2): 439-445 1990) signal sequence and Twin-arginine translocation (Tat) signal sequence (Robisnon, Biol. Chem. 381(2): 89-93. 2000), but not always limited thereto and any signal sequence harboring a high basic N-region can be included.

The hydrophobic fragment of the signal sequence is preferably a peptide composed of 6-21 amino acids containing the 1^{st}-6^{th} amino acids of the signal sequence, but not always limited thereto.

As described above, if one or more amino acids having high pI values like arginine and lysine reside in the N-region, secretional efficiency will be increased. The substitution of amino acids has been well known to those in the art (Sambrook et al., 1989. “Molecular Cloning: A Laboratory Manual”, 2nd ed. Cold Spring Harbor Laboratory Press, Cold Spring Harbor, N.Y.). Mutation in the central characteristic hydrophobic region can be induced with or without mutagenesis of the N-region. The substitution of one or more amino acids in the central characteristic hydrophobic region with another hydrophobic amino acids (for example, phenylalanine, tyrosine, tryptophane, leucine, valine, isoleucine, threonine and alanine) is well-known to those in the art and it is also well understood for those in the art that if the hydropathy profile of the modified signal sequence resulted from the substitution or mutagenesis is similar to the signal sequence of the invention, it might exhibit the similar effect to the signal sequence of the invention.

The secretional enhancer is a polynucleotide encoding a hydrophilic polypeptide composed of at least 60% of hydrophilic amino acids, more preferably composed of at least 70% of hydrophilic amino acids. The length of the polynucleotide is not limited but the polynucleotide encoding a polypeptide comprising 2-50 amino acids is preferred and the polynucleotide encoding a polypeptide comprising 4-25 amino acids is more preferred. At this time, the more preferable number of the amino acids forming a polypeptide for the enhancer is 6-15 and the polynucleotide encoding a polypeptide having a 6 amino acid repeat structure is the most preferred as a secretional enhancer. The hydrophilic amino acids are preferably asparagine, glutamine, serine, lysine, arginine, aspartic acid or glutamic acid, but not always limited thereto, and more preferably lysine or arginine and most preferably a polynucleotide encoding a polypeptide comprising the repeat of 6 strong hydrophilic amino acids such as lysine or arginine. The preferable pI value of the polypeptide encoded by the secretional enhancer of the above is at least 8 and more preferably at least 9 and most preferably at least 10.

In another preferred embodiment of the present invention, the expression vector of the present invention includes an additional polynucleotide encoding a protease recognition site operably linked to the polynucleotide encoding the secretional enhancer (see FIG. 1(i)). The protease recognition site herein is one of factor Xa protease recognition site, enterokinase recognition site, genenase I recognition site or furin recognition site or two or more recognition sites are linked stepwise. And if factor Xa protease is used, the recognition site, Ile-Glu-Gly-Arg is preferred.

In another preferred embodiment of the invention, a polynucleotide encoding the secretional enhancer can be inserted via the SmaI restriction enzyme site (OmpASP fragment-SmaI-Xa) operably linked to the polynucleotide encoding a hydrophobic fragment of a signal sequence or via PCR performed using a primer containing a whole polynucleotide sequence corresponding to the modified signal sequence containing even the entire secretional enhancer. A polynucleotide encoding a target amino acid sequence can be inserted into a secretional enhancer by taking advantage of the SmaI restriction enzyme site.

In a preferred embodiment of the present invention, the expression vector of the invention additionally includes a restriction enzyme site linked to a polynucleotide encoding a secretional enhancer, and through this restriction enzyme site, a gene encoding a heterologous protein can be cloned with ease (see FIG. 1(h)).

In another preferred embodiment of the present invention, the expression vector of the invention additionally includes a gene encoding a heterologous protein operably linked to the above gene construct. The foreign gene can be cloned by the restriction enzyme region and if there is a polynucleotide encoding a protease recognition site inside, the gene is linked in frame with the polynucleotide, so as to secret the heterologous protein and digest with a protease and then produce a native or analog form of the heterologous protein.

The heterologous protein herein is not limited and includes any protein containing one or more of transmembrane domain, transmembrane-like domain or amphipathic domain inside. In such heterologous proteins containing one or more of transmembrane domain, transmembrane-like domain or amphipathic domain, a positively charged region will be attached to the lipid bilayer of the membrane, so the resultant transmembrane-like structure acts as a kind of an anchor to interrupt the periplasmic or extracellular secretion. The expression vector of the present invention is very effective in a periplasmic secretion of those proteins hard to be periplasmically secreted. The expression vector harboring a secretional enhancer of the invention not only is effective in generation of proteins having one or more of transmembrane domain, transmembrane-like domain or amphipathic domain but also increases secretional efficiency of other proteins not containing a transmembrane domain, transmembrane-like domain or amphipathic domain. Therefore, any protein can be produced in soluble form by using the expression vector containing a secretional enhancer of the present invention. As explained herein, the expression vector of the invention is very useful for the production of a protein having one or more of transmembrane domain, transmembrane-like domain or amphipathic domain in soluble form, which seems to be that because when the directional signal is present in the N-terminal of the signal sequence and the hydrophilicity of the modified signal sequence of the invention are higher than those of the internal domain of a heterologous protein, a fusion form of the nascent polypeptide is easily directed to the periplasm. That is, the directionality and hydrophilicity of the modified signal sequence are so higher than the power of the internal domain of the target molecule to attach to the lipid bilayer that secretion is promoted.

The heterologous protein having one or more of transmembrane domain, transmembrane-like domain or amphipathic domain is not limited but olive flounder Hepcidin I is preferably used. If a protein is confirmed by hydropathy profile to have a transmembrane-like domain inside or to have the sequence comprising multiple hydrophilic amino acids serially behind the sequence composed of multiple hydrophobic amino acids, this protein is judged to be the protein having one or more of transmembrane domain, transmembrane-like domain or amphipathic domain, so that it can be applied to the expression system of the invention. And for the judgment, such computer softwares as DNASIS™, DOMpro (Cheng et al., Knowledge Discovery and Data Mining, 13 (1): 1-20, 2006, //www.ics.uci.edu/-baldig/dompro.html), TMpred (//www.ch.embnet.org/software/TMPRED_form.html), HMMTOP (//www.enzim.hu/hmmtop/html/submit.html), TBBpred www.imtech.res.in/raghava/tbbpred/), DAS-TMfilter (//www.enzim.hu/DAS/DAS.html), etc can be used.

The present invention also provides a non-human transformant prepared by transforming a host cell with one of the above expression vectors.

The host cell herein is not limited, but a prokaryotic cell or a eukaryotic cell is preferred. The prokaryotic cell is preferably selected from a group consisting of virus, E. coli, and Bacillus, but not always limited thereto. The eukaryotic cell is preferably selected from mammalian cells, insect cells, yeasts and plant cells, but not always limited thereto.

The present invention further provides a method for improving secretional efficiency of a heterologous protein comprising the following steps:

1) Analyzing the hydropathy profile of a heterologous protein;

2) Judging whether the heterologous protein analyzed in step 1) contains one or more of transmembrane domain, transmembrane-like domain or amphipathic domain inside;

3) (a) Constructing a gene construct composed of polynucleotides encoding a fusion protein in which the heterologous protein is combined with a polypeptide fragment containing the N-region of a signal sequence or a fusion protein in which the heterologous protein is combined with a polypeptide fragment containing the N-region of a signal sequence and a protease recognition site, when the heterologous protein is confirmed not to contain a transmembrane domain, transmembrane-like domain or amphipathic domain in step 2), and

(b) Constructing a gene construct composed of polynucleotides encoding a fusion protein containing a hydrophobic fragment comprising the N-region and central characteristic hydrophobic region of a signal sequence, a secretional enhancer and the heterologous protein sequentially or a fusion protein containing a hydrophobic fragment comprising the N-region and central characteristic hydrophobic region of a signal sequence, a secretional enhancer, a protease recognition site and the heterologous protein sequentially, when the heterologous protein is confirmed to have one or more of transmembrane domain, transmembrane-like domain and amphipathic domain in step 2);

4) Constructing a recombinant expression vector by inserting the gene construct prepared in step 3) operably into an expression vector;

5) Constructing a transformant by transforming a host cell with the recombinant expression vector of step 4); and

6) Culturing the transformant of step 5).

Herein, the heterologous protein is not limited and any protein that is acceptable for those in the art can be used. For example, a protein selected from a group consisting of an antigen, an antibody, a cell receptor, an enzyme, a structural protein, a serum, and a cell protein is preferred and a protein that is expressed in insoluble form is more preferred. In a preferred embodiment of the present invention, Mefp1 multimer and olive flounder Hepcidin I were used as a heterologous protein, but not always limited thereto.

The hydropathy profile herein is preferably analyzed by computer softwares or web-based applications for hydropathy profile analysis, but not always limited thereto. And the computer software for the analysis is selected from a group consisting of DNASIS™ (Hitachi, Japan), Visual OMP (DNA software, USA), Lasergene (DNASTAR, USA), pDPAW32 (USA) and NetSupport DNA (NetSupport Inc. USA) and among these DNASIS™ (Hitachi, Japan) is more preferred.

The secretional enhancer is preferably a hydrophilic polypeptide containing hydrophilic amino acids by at least 60% and more preferably at least 70%, but not limited thereto. The length of the polypeptide is not limited but preferably 2-50 amino acids long and more preferably 4-25 and most preferably 6-15 amino acids long. Particularly, the polypeptide is most preferably composed of the repeat of 6 hydrophilic amino acids. The preferable pI value of the hydrophilic polypeptide used as a secretional enhancer is at least 8, more preferable pI value is at least 9 and most preferable pI value is at least 10, but not always limited thereto.

The hydrophilic amino acid hereinabove is not limited but preferably asparagine, glutamine, serine, lysine, arginine, aspartic acid or glutamic acid and more preferably lysine or arginine.

In a preferred embodiment of the present invention, a protease recognition site is additionally inserted in between a secretional enhancer and a heterologous protein.

The host cell of the invention is not limited but preferably a prokaryotic or a eukaryotic cell. The prokaryotic cell is not limited but preferably selected from a group consisting of virus, E. coli, and Bacillus. The eukaryotic cell is not limited but preferably selected from a group consisting of mammalian cells, insect cells, yeasts and plant cells.

The present invention also provides a method for preparing a fusion heterologous protein comprising the following steps:

1) Analyzing hydropathy profile of a heterologous protein;

2) Judging whether the heterologous protein analyzed in step 1) contains one or more of transmembrane domain, transmembrane-like domain or amphipathic domain inside;

3) (a) Constructing a gene construct composed of polynucleotides encoding a fusion protein in which the heterologous protein is combined with a polypeptide fragment containing the N-region of a signal sequence and a protease recognition site, when the heterologous protein is confirmed not to contain a transmembrane domain, transmembrane-like domain or amphipathic domain in step 2) and

(b) Constructing a gene construct composed of polynucleotides encoding a fusion heterologous protein containing a hydrophobic fragment comprising the N-region and central characteristic hydrophobic region of a signal sequence, a secretional enhancer, a protease recognition site and a heterologous protein sequentially, when the heterologous protein is confirmed to have one or more of transmembrane domain, transmembrane-like domain and amphipathic domain in step 2);

4) Constructing a recombinant expression vector by inserting the gene construct prepared in step 3) operably into an expression vector;

5) Constructing a transformant by transforming a host cell with the recombinant expression vector of step 4);

6) Culturing the transformant of step 5); and

7) Separating a fusion heterologous protein from the culture solution of step 6).

Herein, the heterologous protein is not limited and any protein that is acceptable for those in the art can be included, which is preferably selected from a group consisting of an antigen, an antibody, a cell receptor, an enzyme, a structural protein, a serum, and a cell protein and particularly a protein that is expressed in insoluble form is more preferred. In a preferred embodiment of the present invention, Mefp1 multimer and olive flounder Hepcidin I were used as a heterologous protein, but not always limited thereto.

The hydropathy profile herein is preferably analyzed by computer softwares or web-based applications for hydropathy profile analysis, but not always limited thereto. And the computer software for the analysis is selected from a group consisting of DNASIS™ (Hitachi, Japan), Visual OMP (DNA software, USA), Lasergene (DNASTAR, USA), pDPAW32 (USA) and NetSupport DNA (NetSupport Inc. USA) and among these DNASIS™ (Hitachi, Japan) is more preferred.

The secretional enhancer is preferably a hydrophilic polypeptide containing hydrophilic amino acids by at least 60% and more preferably at least 70%, but not limited thereto. The length of the polypeptide is not limited but preferably 2-50 amino acids long and more preferably 4-25 and most preferably 6-15 amino acids long. Particularly, the polypeptide is most preferably composed of the repeat of 6 hydrophilic amino acids. The preferable pI value of the hydrophilic polypeptide used as a secretional enhancer is at least 8, more preferable pI value is at least 9 and most preferable pI value is at least 10, but not always limited thereto.

The hydrophilic amino acid hereinabove is not limited but preferably asparagine, glutamine, serine, lysine, arginine, aspartic acid or glutamic acid and more preferably lysine or arginine.

The host cell of the invention is not limited but preferably a prokaryotic or a eukaryotic cell. The prokaryotic cell is not limited but preferably selected from a group consisting of virus, E. coli, and Bacillus. The eukaryotic cell is not limited but preferably selected from a group consisting of mammalian cells, insect cells, yeasts and plant cells.

The protein expressed in the transformant transformed with the said expression vector is recovered, resulting in the production of the target fusion protein. The recovery is performed by the conventional method well known to those in the art.

Herein, the heterologous protein is not limited and any protein that is acceptable for those in the art can be used. For example, a protein selected from a group consisting of an antigen, an antibody, a cell receptor, an enzyme, a structural protein, a serum, and a cell protein is preferred and a protein that is expressed in insoluble form is more preferred. In a preferred embodiment of the present invention, Mefp1 multimer and olive flounder Hepcidin I were used as a heterologous protein, but not always limited thereto.

If a therapeutic protein targeting brain disease, for example β-amyloid specific scFv (single-chain variable fragment) is used as a heterologous protein herein, the resultant fusion protein of the modified signal sequence of the invention and the inserted heterologous protein can pass through the blood-brain barrier to be effective directly in the brain, which is not expected from the conventional protein. Therefore, the method of the present invention greatly contributes to drug delivery system, in particular for the treatment of brain disease. Not only passing through the blood-brain barrier, the recombinant fusion heterologous protein of the invention can pass through the stomach wall before being decomposed when it is orally administered or can pass through the skin so as to be delivered safely inside of a body when it is applied by spray or patch. Therefore, the fusion protein of the invention overcomes the problem of the conventional method which is limited in the administration pathway (intravenous injection, intramuscular injection, hypodermic injection or nasal administration), and further facilitates more simple and comfortable administrations including oral administration and transdermal administration.

The present invention also provides a recombinant fusion heterologous protein according to the above method.

The heterologous protein herein is not limited but a therapeutic protein targeting brain disease is preferred. The recombinant fusion protein prepared by the method above can have a transmembrane region through which it can pass through blood-brain barrier, because it contains the modified signal sequence of the invention.

The present invention further provides a pharmaceutical composition containing a fusion protein composed of the modified signal sequence and a heterologous protein prepared by the above method and a pharmaceutically acceptable carrier. The pharmaceutical composition can be used for the treatment of brain disease, but not always limited thereto.

The present invention also provides a method for preparing the native form of a heterologous protein comprising the following steps:

1) Analyzing hydropathy profile of a heterologous protein;

2) Judging whether the heterologous protein analyzed in step 1) contains one or more of transmembrane domain, transmembrane-like domain or amphipathic domain inside;

3) (a) Constructing a gene construct composed of polynucleotides encoding a fusion protein in which the heterologous protein is combined with a polypeptide fragment containing the N-region of a signal sequence and a protease recognition site, when the heterologous protein is confirmed not to contain a transmembrane domain, transmembrane-like domain or amphipathic domain in step 2), and
(b) Constructing a gene construct composed of polynucleotides encoding a fusion heterologous protein containing a hydrophobic fragment comprising the N-region and central characteristic hydrophobic region of a signal sequence, a secretional enhancer, a protease recognition site and a heterologous protein sequentially, when the heterologous protein is confirmed to have one or more of transmembrane domain, transmembrane-like domain and amphipathic domain in step 2);

4) Constructing a recombinant expression vector by inserting the gene construct prepared in step 3) operably into an expression vector;

5) Constructing a transformant by transforming a host cell with the recombinant expression vector of step 4);

6) Culturing the transformant of step 5); and

7) Separating a fusion heterologous protein from the culture solution of step 6); and

8) Separating the native form of the heterologous protein from the fusion protein separated in step 7) after digesting the protease recognition site with a protease.

Herein, the heterologous protein is not limited and any protein that is acceptable for those in the art can be used. For example, a protein selected from a group consisting of an antigen, an antibody, a cell receptor, an enzyme, a structural protein, a serum, and a cell protein is preferred and a protein that is expressed in insoluble form is more preferred. In a preferred embodiment of the present invention, Mefp1 multimer and olive flounder Hepcidin I were used as a heterologous protein, but not always limited thereto.

The hydropathy profile herein is preferably analyzed by computer softwares or web-based applications for hydropathy profile analysis, but not always limited thereto. And the computer software for the analysis is selected from a group consisting of DNASIS™ (Hitachi, Japan), Visual OMP (DNA software, USA), Lasergene (DNASTAR, USA), pDPAW32 (USA) and NetSupport DNA (NetSupport Inc. USA) and among these DNASIS™ (Hitachi, Japan) is more preferred. As a web-based application, an application provided by Innovagen Inc. (Sweden) through its home-page (//www.innovagen.se/custom-peptide-synthesis/peptide-property-calculator/peptide-property-calculator.asp) can be used.

The secretional enhancer is preferably a hydrophilic polypeptide containing hydrophilic amino acids by at least 60% and more preferably at least 70%, but not limited thereto. The length of the polypeptide is not limited but preferably 2-50 amino acids long and more preferably 4-25 and most preferably 6-15 amino acids long. Particularly, the polypeptide is most preferably composed of the repeat of 6 hydrophilic amino acids. The preferable pI value of the hydrophilic polypeptide used as a secretional enhancer is at least 8, more preferable pI value is at least 9 and most preferable pI value is at least 10, but not always limited thereto.

The hydrophilic amino acid hereinabove is not limited but preferably asparagine, glutamine, serine, lysine, arginine, aspartic acid or glutamic acid and more preferably lysine or arginine.

In another preferred embodiment of the present invention, a protease recognition site is additionally inserted in between the secretional enhancer and the foreign protein.

The host cell of the invention is not limited but preferably a prokaryotic or a eukaryotic cell. The prokaryotic cell is not limited but preferably selected from a group consisting of virus, E. coli, and Bacillus. The eukaryotic cell is not limited but preferably selected from a group consisting of mammalian cells, insect cells, yeasts and plant cells.

The protein expressed in the transformant transformed with the said expression vector is recovered, resulting in the production of the target fusion protein. The recovery is performed by the conventional method well known to those in the art. The native form of the heterologous protein can be separated from the fusion protein by treating a protease facilitating the cut of the inserted protease recognition site off from the fusion heterologous protein. The protease herein is preferably factor Xa, enterokinase, genenase I and furin, but not always limited thereto. In the meantime, if factor Xa protease is used, the recognition site of the amino acid sequence is preferably Ile-Glu-Gly-Arg.

In a preferred embodiment of the present invention, the present invention provides a method for improving secretional efficiency comprising the following steps:

1) Constructing a recombinant expression vector by operably linking a gene encoding a heterologous protein to the restriction enzyme site of the expression vector of the invention;

2) Generating a transformant by transforming a host cell with the recombinant expression vector of step 1); and

3) Culturing the transformant of step 2).

Herein, the host cell is not limited but preferably a prokaryotic or a eukaryotic cell. The prokaryotic cell is not limited but preferably selected from a group consisting of virus, E. coli, and Bacillus. The eukaryotic cell is not limited but preferably selected from a group consisting of mammalian cells, insect cells, yeasts and plant cells.

The present invention also provides a screening method for a secretional enhancer improving secretion of a heterologous protein, which comprises the following steps:

1) Constructing an expression vector containing a gene construct in which a promoter, a polynucleotide encoding a polypeptide fragment containing the N-region of a signal sequence or a hydrophobic fragment containing the N-region and central characteristic hydrophobic region of a signal sequence, a restriction enzyme site for the insertion of a secretional enhancer candidate and a polynucleotide encoding a heterologous protein are operably linked to one another;

2) Constructing a recombinant expression vector by inserting a polynucleotide encoding a secretional enhancer candidate sequence comprising hydrophilic amino acids into the restriction enzyme site of the expression vector;

3) Generating a transformant by transforming a host cell with the recombinant expression vector of step 2);

4) Culturing the transformant of step 3);

5) Measuring the expression level of the heterologous protein in soluble fractions or culture solutions of both the transformant (control) transformed with the expression vector of step 1) and the transformant of step 4); and

6) Selecting a secretional enhancer which significantly increases the expression level of the heterologous protein inserted, compared with a control.

### BEST MODE

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Cloning of an Adhesive Protein Gene DNA Multimer Cassette

The present inventors prepared a synthetic mefp1 DNA based on the basic unit of the Mefp1 amino acid sequence represented by SEQ. ID. NO: 1 (Ala Lys Pro Ser Tyr Pro Pro Thr Tyr Lys) by using a forward primer represented by SEQ. ID. NO: 2 (5′-TAC AAA GCT AAG CCG TCT TAT CCG CCA ACC-3′) and a reverse primer represented by SEQ. ID. NO: 3 (5′-TTT GTA GGT TGG CGG ATA AGA CGG CTT AGC-3′). For the left adaptor (referred as “La” hereinafter) synthetic DNA (contains BamHI/EcoRI/SmaI), a forward primer represented by SEQ. ID. NO: 4 (5′-GAT CCG AAT TCC CCG GG-3′) and a reverse primer represented by SEQ. ID. NO: 5 (5′-TTT GTA CCC GGG GAA TTC G-3′) were used. For the right adaptor (referred as “Ra” hereinafter) synthetic DNA (contains Arg/HindIII/SalI/XhoI), a forward primer represented by SEQ. ID. NO: 6 (5′-TAC AAA CGT AAG CTT GTC GAC C-3′) and a reverse primer represented by SEQ. ID. NO: 7 (5′-TCG AGG TCG ACA AGC TTA CG-3′) were used. Thereafter, mefp1 DNA multimer was constructed by the method described in Korean Patent No. 379,025, which was then cloned into the vector pBluescriptIISK(+) (Stratagene, USA). Screening for transformants yielded a construct containing the left adaptor (La) sequence, seven mefp1 DNA repeats and the Ra sequence was performed and the screened construct was named as pBluescriptIISK(+)La-7×mefp1-Ra (FIG. 2).

TABLE 1

Primers, plasmid clones and the expression of the
recombinant Mefp1

Clones constructed in

| | | |
|---|---|---|
| SEQ. | pET22b (+) containing | Mefp1 |

| ID. | | the whole and a part of | expression | | |
|---|---|---|---|---|---|
| NO: | Primer sequence | OmpASB thereof | T | S | P |

Forward primers containing various lengths of OmpASB-Mefp1

| | | | | | |
|---|---|---|---|---|---|
| 8 | [symbol] | pET22b (+) ompASP₁₋₃- | + | + | + |
| | AAG CCG TCT TAT CCG | 7 × mefp1* | | | |
| | CCA ACC | | | | |
| 9 | [symbol] | pET22b (+) ompASP₁₋₄- | + | + | + |
| | GCT AAG CCG TCT TAT | 7 × mefp1* | | | |
| | CCG CCA ACC | | | | |
| 10 | [symbol] | pET22b (+) ompASP₁₋₅- | + | + | + |
| | [symbol] | 7 × mefp1* | | | |
| | TAT CCG CCA ACC | | | | |
| 11 | [symbol] | pET22b (+) ompASP₁₋₆- | + | + | + |
| | [symbol] | 7 × mefp1* | | | |
| | TCT TAT CCG CCA ACC | | | | |
| 12 | [symbol] | pET22b (+) ompASP₁₋₇- | + | + | + |
| | [symbol] | 7 × mefp1* | | | |
| | CCG TCT TAT CCG CCA | | | | |
| | ACC | | | | |
| 13 | [symbol] | pET22b (+) ompASP₁₋₈- | + | + | + |
| | [symbol] | 7 × mefp1* | | | |
| | AAG CCG TCT TAT CCG | | | | |
| | CCA ACC | | | | |
| 14 | [symbol] | pET22b (+) ompASP₁₋₉- | + | + | + |
| | [symbol] | 7 × mefp1* | | | |
| | GCT AAG CCG TCT TAT | | | | |
| | CCG CCA ACC | | | | |
| 15 | [symbol] | pET22b (+) ompASP₁₋₁₀- | + | + | + |
| | [symbol] | 7 × mefp1* | | | |
| | [symbol] | | | | |
| | TAT CCG CCA ACC | | | | |
| 16 | [symbol] | pET22b (+) ompASP₁₋₁₁- | + | + | + |
| | [symbol] | 7 × mefp1* | | | |
| | [symbol] | | | | |
| | TCT TAT CCG CCA ACC | | | | |
| 17 | [symbol] | pET22b (+) ompASP₁₋₁₃- | + | + | + |
| | [symbol] | 7 × mefp1* | | | |
| | [symbol] | | | | |
| | AAG CCG TCT TAT CCG | | | | |
| | CCA ACC | | | | |
| 18 | [symbol] | pET22b (+) ompASP₁₋₁₅- | + | + | + |
| | [symbol] | 7 × mefp1* | | | |
| | [symbol] | | | | |
| | [symbol] | | | | |
| | TAT CCG CCA ACC | | | | |
| 19 | [symbol] | pET22b (+) ompASP₁₋₂₁- | + | + | + |
| | [symbol] | 7 × mefp1* | | | |
| | [symbol] | | | | |
| | [symbol] | | | | |
| | [symbol] | | | | |
| | TCT TAT CCG CCA ACC | | | | |
| 20 | [symbol] | pET22b (+) ompASP₁₋₂₃- | + | + | + |
| | [symbol] | 7 × mefp1* | | | |
| | [symbol] | | | | |
| | [symbol] | | | | |
| | [symbol] | | | | |
| | AAG CCG TCT TAT CCG | | | | |
| | CCA ACC | | | | |
| 21 | [symbol] | pET22b (+) ompASP₁₋₈- | + | + | + |
| | [symbol] | Xa-7 × mefp1* | | | |
| | GAA GGT CGT GCT AAG | | | | |
| | CCG TCT TAT CCG CCA | | | | |
| | ACC | | | | |
| 22 | [symbol] | pET22b (+) ompASP₁₋₈- | + | + | + |
| | [symbol] | SmaI-Xa-7 × mefp1* | | | |
| | GGG ATC GAA GGT CGT | | | | |
| | GCT AAG CCG TCT TAT | | | | |
| | CCG CCA ACC | | | | |

Reverse primer

| | | |
|---|---|---|
| 23 | CTC GAG GTC GAC AAG | No corresponding clone |
| | CTT ACG | |

Thick Italic letters: indicate various sized oligonucleotides of the whole and a part of OmpASP.

Thick letters: oligonucleotides of the SmaI site.

Underlined thick letters: oligonucleotides of the factor Xa recognition site.

General letters: oligonucleotides of Mefp1 region shown in FIG. 2.

Reverse primer: complementary oligonucleotide sequences to Ra (right adapter; Arg/HindIII/SalI/XhoI) shown in FIG. 2.

OmpA signal peptide (OmpASP) is composed of 23 amino acid residues (MKKTAIAIAVALAGFATVAQAAP: SEQ. ID. NO: 46) (Movva et al., J. Biol. Chem. 255, 27-29, 1980).

*: surplus sequences of Ra and His tag (6×His).

mefp1: Mefp1 gene

Abbreviations: T-total protein; S-soluble fraction; and P-periplasm fraction.

Expression of recombinant Mefp1 protein: “−”; no-expression, “+”; expression.

TABLE 2
pI value, hydrophobicity average value and expression of
the soluble recombinant Mefp1 protein according to the
length of OmpASP

| OmpASP and its | | Hopp & | Expression of the |
|---|---|---|---|
| segments of | | Woods scale | soluble |
| various lengths | pI | hydrophobicity | recombinant Mefp1 |
| OmpASP₁ | 5.70 | — | NT |
| OmpASP₁₋₂ | 9.90 | — | NT |
| OmpASP₁₋₃ | 10.55 | — | + |
| OmpASP₁₋₄ | 10.55 | — | + |
| OmpASP₁₋₅ | 10.55 | — | + |
| OmpASP₁₋₆ | 10.55 | −0.03 | + |
| OmpASP₁₋₇ | 10.55 | −0.09 | + |
| OmpASP₁₋₈ | 10.55 | −0.31 | + |
| OmpASP₁₋₉ | 10.55 | −0.33 | + |
| OmpASP₁₋₁₀ | 10.55 | −0.44 | + |
| OmpASP₁₋₁₁ | 10.55 | −0.45 | + |
| OmpASP₁₋₁₂ | 10.55 | −0.56 | NT |
| OmpASP₁₋₁₂ | 10.55 | −0.56 | + |
| OmpASP₁₋₁₄ | 10.55 | −0.52 | NT |
| OmpASP₁₋₁₅ | 10.55 | −0.65 | + |
| OmpASP₁₋₂₁ | 10.55 | −0.61 | + |
| OmpASP₁₋₂₃ | 10.55 | −0.58 | + |

OmpASP length dependent pI value and hydrophobicity (Hopp & Woods scale with window size: 6 and threshold line: 0.00) were calculated by DNASIS™. The Hopp and Woods scale hydrophobicity represents that ‘−’ indicates no value, whereas the ‘− value’ indicates hydrophobic. As absolute value increases, hydrophobicity increases. Expression of recombinant Mefp1 protein: ‘NT’; not tested, ‘+’; expression.

### EXAMPLE 2

### Expression of an Adhesive Protein mefp1

In the previous study, Mefp1 expressed an insoluble inclusion body when Met-Mefp1 was used as a leader sequence (Kitamura et al., J Polym. Sci. Ser. A 37:729-736, 1999). The present inventors introduced the signal sequence OmpASP (OmpA signal peptide) to induce expression of a target protein in soluble form, for which PCR was performed using the mefp1 sequence of FIG. 2 as a template to construct a clone harboring different sizes of ompASP and the mefp1 cassette (Table 1).

Transformants of E. coli BL21(DE3) generated by using the expression vector containing the signal sequence shown in Table 1 were cultured in LB medium (tryptone 20 g, yeast extract 5.0 g, NaCl 0.5 g, KCl 1.86 mg/l) in the presence of 50 μg/ml of ampicillin at 30° C. for 16 hours. The culture solution was diluted 200-fold with LB medium. The diluted culture solution was incubated to reach OD₆₀₀ of 0.3 and then IPTG was added to a final concentration of 1 mM. The culture solution was incubated for further 3 hours for expression. Then, 1 ml of the culture solution was centrifuged at 4° C. for 30 minutes with 4,000×g and pellet was resuspended in 100-200 μl of sample buffer (0.05 M Tris-HCl, pH 6.8, 0.1 M DTT, 2% SDS, 1% glycerol, 0.1% bromophenol blue). The resuspension was disrupted by sonication using 100 3-s pulses to release the total proteins and the insoluble fraction was separated by centrifugation at 4° C. with 16,000 rpm for 30 minutes to eliminate cell debris. To prepare periplasmic fractions, induced cells were subjected to osmotic shock (Nossal and Heppel, J. Biol. Chem. 241:3055-3062, 1966). The lysate of total proteins, the soluble fraction, and the periplasmic fraction were separated using 16% SDS-PAGE (Laemmli, Nature 227:680-685, 1970) and visualized using Coomassie brilliant blue stain (Sigma, USA). The gel obtained from SDS-PAGE was transferred to a nitrocellulose membrane (Roche, USA). After blocking with 5% skim milk (Difco, USA), the membrane was incubated in a solution containing 0.4 μg/ml anti-His6 monoclonal antibody (Santa Cruz Biotechnology, USA) for 2 hours at 37° C. Horseradish peroxidase (HRP) conjugated rabbit anti-mouse IgG (Santa Cruz Biotechnology, USA) was used as the secondary antibody and 3,3′-diaminobenzidine tetrahydrochloride (DAB, Sigma, USA) was used as the staining substrate.

As a result, all of the OmpA signal peptides from the leader sequence OmpASP₁₋₃ to OmpASP₁₋₂₃ tested herein successfully directed the expression of soluble periplasmic Mefp1 (Table 1 and FIG. 3). It was also confirmed that what directs the expression of Mefp1 in soluble form is not the full length of OmpASP₁₋₂₃ but the fraction of OmpASP₁₋₃, which is only OmpASP₁₋₃ is necessary to direct Mefp1 precursor to the periplasm. The expression level was not associated with the length of a leader sequence and no evidence for the presence of a secretional enhancer was found in the central characteristic hydrophobic region (OmpASP₇₋₁₄) and the C-region ending with a cleavage site (OmpASP₁₅₋₂₃). pI value and the Hopp & Woods scale hydrophobicity of the signal sequence of OmpASP with different length were analyzed. As a result, all the sequences from OmpASP₁₋₃ to OmpASP₁₋₂₃ had an equal pI value, which was 10.55, but the Hopp & Woods scale hydrophobicity values were diverse (Table 2). The constant pI value is the most important factor in the functioning of OmpASP fragments as directional signals for soluble protein expression.

### EXAMPLE 3

### Production of the Native Form of an Adhesive Protein mefp1

To produce Mefp1 with its native N-terminus, the present inventors performed PCR using pBluescriptIISK(+)-La-7×mefp1-Ra (FIG. 2) as a template and a synthetic oligonucleotide encoding the OmpASP₁₋₈-Xa-Mefp1 containing factor Xa cleavage site for cleaving the C-terminal end as a forward primer to construct pET-22b(+)(ompASP₁₋₈-Xa-7×mefp1*) (*: Ra-6×His, Ra derived from the right adaptor; 6×His derived from His tag) clone, based on the result of soluble expression by the shortened OmpASP (Table 1). The constructed vector was tested for the expression by the transformation and Western blotting as described in Example 2.

As a result, this clone produced soluble protein OmpASP₁₋₈-Xa-7×Mefp1*. Further, the 7×Mefp1* protein with a native amino acid terminus was obtained by the removal of the OmpASP₁₋₈-Xa sequence with factor Xa protease (FIG. 4).

To modify the signal sequence region of the above clone conveniently, the present inventors introduced a SmaI site into the signal sequence to construct pET-22 γ(+)(ompASP₁₋₈-SmaI-Xa-7×mefp1*) clone by PCR (Table 1) in order to maintain the same copy number of target gene cassette against the various copy of mefp1 usually obtained from the repeated mefp1 template by PCR. The resulting OmpASP₁₋₈-Sma I-Xa-7×Mefp1* was digested with factor Xa protease to cleave off the OmpASP₁₋₈-Sma I-Xa and the obtained protein was confirmed to be 7×Mefp1* with a native amino terminus. By inserting up to six homologous amino acid codons in the SmaI site of pET-22b(+) (ompASP₁₋₈-Sma I-Xa-7×mefp1*), it was confirmed that the hydrophilic amino acids Arg and Lys slightly increased the level of expression.

### EXAMPLE 4

### Investigation on the Function of the Adhesive Protein Mefp1

Mefp1 expressed from the pET-22b(+) (ompASP₁₋₈-Xa-7×mefp1*) clone was separated as follows. The induced cells were centrifuged at 4° C. for 30 minutes with 4,000×g. The supernatant was removed and pellet was washed and frozen at −70° C. or suspended in PBS (pH 8.0), followed by sonication using a sonicator. The lysed cells were centrifuged at 4° C. for 30 minutes with 12,000×g. The supernatant was treated with a protease factor Xa (New England Biolabs, USA) to cut off the signal sequence OmpASP₁₋₈-Xa, which was then filtered through a 0.45 μm syringe filter. The native Mefp1 protein (7×Mefp1*) was purified by His tag purification kit (Qiagen, USA) according to the manufacturer's instructions. 1 ml of Ni²⁺ chelating resin was equilibrated with 5 ml of distilled water, 3 ml of 50 mM NiSO₄, and 5 ml of 1× binding buffer (50 mM NaCl, 20 mM Tris-HCl, 5 mM imidazole, pH 7.9). The supernatant was loaded on the column and washed with 10 ml of 1× binding buffer and 6 ml of washing buffer (60 mM imidazole in PBS). The protein of interest was eluted with 6 ml of elution buffer (1,000 mM imidazole in PBS) and the eluted fractions were analyzed by 12% SDS-PAGE.

The functions of the recombinant Mefp1 with a native amino terminus were investigated. Protein samples were resolved in 5% acetic acid buffer (Hwang et al., Appl. Environ. Microbiol. 70:3352-3359, 2004) and tyrosinase (tyrosinase; Sigma, USA) was used to transform tyrosine into DOPA. Prior to adhesion assay, 1 mg/ml of protein was modified with 10 U of tyrosinase at room temperature for 6 hours with shaking. BSA in 5% acetic acid buffer was used as a non-adhesive protein control.

As a result, compared with BSA used as a control, the rcombinant Mefp1 protein (7×Mefp1*) with a native amino terminus exhibited significant cohesiveness (FIG. 5). Therefore, the soluble recombinant Mefp1 protein produced by the method of the invention was confirmed to have a proper structure and an original protein function.

### EXAMPLE 5

### Screening of a Secretional Enhancer for the Expression of a Soluble Olive Flounder Hepcidin 1

As the above Example 2, the present inventors expressed olive flounder Hepcidin I (Kim et al., Biosci. Biotechnol. Biochem. 69, 1411-1414, 2005) as a fusion protein with various lengths of OmpASP by the same manner as used for the expression of Mefp1 but the fusion protein was not expressed in soluble form (Table 3). Sequence of olive flounder Hepcidin I is as follows (SEQ. ID. NO: 47):

His Ile Ser His Ile Ser Met Cys Arg Trp Cys Cys Asn Cys Cys Lys Ala Lys Gly Cys Gly Pro Cys Cys Lys Phe.

The present inventors presumed that the presence of four disulfide bonds and one amphipathic domain in olive flounder Hepcidin I was the reason why the fusion protein OmpASPₜᵣ-olive flounder Hepcidin I could not be expressed in soluble form as effectively as Mefp1 having a plain structure (pI: 10.03; hydrophobicity: −0.05).

To screen a secretional enhancer for soluble protein expression, the present inventors constructed pET-22b(+)[ompASP₁₋₁₀-( )-Xa-ofhepcidinI**] (Table 3) by modifying the signal sequence as a form of OmpASP₁₋₁₀-( )-Xa, in which the N-terminal region of the signal sequence was set as OmpASP₁₋₁₀ and the 6 homologous sequence of six amino acids such as arginine, lysine, glutamic acid, aspartic acid, tyrosine, phyenylalanine and tryptophan affecting pI value and hydrophobicity/hydrophilicity value were added to -( )- to change the C-terminal -( )-Xa region (Table 4), followed by investigation of the expression of soluble olive flounder Hepcidin I. As a result, the hydrophilic amino acids Arg and Lys increased the expression level of soluble Hepcidin I but the clones without these amino acids exhibited weak expression of soluble Hepcidin I (FIG. 6). The above results indicate that these amino acids arginine and lysine attached at the C-terminal of the signal peptide moiety function as a strong secretional enhancer because of their high pI and hydrophilicity, while other amino acids function as a comparatively weak secretional enhancer (FIG. 6 and Table 4). Therefore, the amino acid additioned to the C-terminal of the modified signal sequence increases the secretional efficiency because of the high pI and hydrophilicity of the added amino acids.

TABLE 3

Primers, plasmid clones and the expression of olive
flounder Hepcidin I

| | | |
|---|---|---|
| | Clones | |
| | constructed | Expression |
| | in pET22b | of olive |
| SEQ. | (+) containing | flounder |

| ID. | | OmpA signal | Hepcidin I | | |
|---|---|---|---|---|---|
| NO: | Primer sequence | peptide fragment | T | S | P |

Forward primer

| | | | | | |
|---|---|---|---|---|---|
| 24 | CAT ATG AAA AAG ACA | pET22b (+) ompASP₁₋₄ | − | − | − |
| | CAC ATC AGC CAC ATC | ofhepI** | | | |
| | TCC ATG TGC | | | | |
| 25 | CATATG AAA AAG ACA | pET22b (+) ompASP₁₋₆ | + | − | − |
| | GCT ATC CAC ATC AGC | ofhepI** | | | |
| | CAC ATC TCC ATG TGC | | | | |
| 26 | CAT ATG AAA AAG ACA | pET22b (+) ompASP₁₋₈ | + | − | − |
| | GCT ATC GCG ATTCAC | ofhepI** | | | |
| | ATC AGC CAC ATC TCC | | | | |
| | ATG TGC | | | | |
| 27 | CAT ATG AAA AAG ACA | pET22b (+) ompASP₁₋₁₀ | + | − | − |
| | GCT ATC GCG ATT GCA | ofhepI** | | | |
| | GTG CAC ATC AGC CAC | | | | |
| | ATC TCC ATG TGC | | | | |
| 28 | CAT ATG AAA AAG ACA | pET22b (+) ompASP₁₋₁₂ | + | − | − |
| | GCT ATC GCG ATT GCA | ofhepI** | | | |
| | GTG GCA CTG CAC ATC | | | | |
| | AGC CAC ATC TCC ATG | | | | |
| | TGC | | | | |
| 30 | CAT ATG AAA AAG ACA | pET22b (+) ompASP₁₋₁₀- | + | + | + |
| | GCT ATC GCG ATT GCA | 6 × Arg-Xa-ofhepI** | | | |
| | [symbol] | | | | |
| | [symbol] | | | | |
| | CGT) CAC ATC AGC CAC | | | | |
| | ATC TCC ATG TGC | | | | |
| 31 | CAT ATG AAA AAG ACA | pET22b (+) ompASP₁₋₁₀- | + | + | + |
| | GCT ATC GCG ATT GCA | 6 × Lys-Xa-ofhepI** | | | |
| | [symbol] | | | | |
| | [symbol] | | | | |
| | CGT) CAC ATC AGC CAC | | | | |
| | ATC TCC ATG TGC | | | | |
| 32 | CAT ATG AAA AAG ACA | pET22b (+) ompASP₁₋₁₀- | + | +/− | +/− |
| | GCT ATC GCG ATT GCA | 6 × Glu-Xa-ofhepI** | | | |
| | [symbol] | | | | |
| | GAA GAG (ATC GAA GGT | | | | |
| | CGT) CAC ATC AGC CAC | | | | |
| | ATC TCC ATG TGC | | | | |
| 33 | CATATG AAA AAG ACA | pET22b (+) ompASP₁₋₁₀- | + | +/− | +/− |
| | GCT ATC GCG ATT GCA | 6 × Asp-Xa-ofhepI** | | | |
| | [symbol] | | | | |
| | [symbol] | | | | |
| | CGT) CAC ATC AGC CAC | | | | |
| | ATC TCC ATG TGC | | | | |
| 34 | CAT ATG AAA AAG ACA | pET22b (+) ompASP₁₋₁₀- | + | +/− | +/− |
| | GCT ATC GCG ATT GCA | 6 × Tyr-Xa-ofhepI** | | | |
| | [symbol] | | | | |
| | [symbol] | | | | |
| | CTG) CAC ATC AGC CAC | | | | |
| | ATC TCC ATG TGC | | | | |
| 35 | CAT ATG AAA AAG ACA | pET22b (+) ompASP₁₋₁₀- | + | +/− | +/− |
| | GCT ATC GCG ATT GCA | 6 × Phe-Xa-ofhepI** | | | |
| | [symbol] | | | | |
| | [symbol] | | | | |
| | CGT) CAC ATC AGC CAC | | | | |
| | ATC TCC ATG TGC | | | | |
| 29 | CAT ATG AAA AAG ACA | pET22b (+) ompASP₁₋₁₀- | + | +/− | +/− |
| | GCT ATC GCG ATT GCA | 6 × Trp-Xa-ofhepI** | | | |
| | [symbol] | | | | |
| | [symbol] | | | | |
| | CGT) CAC ATC AGC CAC | | | | |
| | ATC TCC ATG TGC | | | | |
| 36 | CAT ATG AAA AAG ACA | pET22b (+) ompASP₁₋₆- | + | + | + |
| | [symbol] | 6 × Arg-Xa-ofhepI** | | | |
| | [symbol] | | | | |
| | GGT CGT) CAC ATC AGC | | | | |
| | CAC ATC TCC ATG TGC | | | | |
| 37 | CAT ATG AAA AAG ACA | pET22b (+) ompASP₁₋₈- | + | + | + |
| | [symbol] | 6 × Arg-Xa-ofhepI** | | | |
| | [symbol] | | | | |
| | (ATC GAA GGT CGT) | | | | |
| | CAC ATC AGC CAC ATC | | | | |
| | TCC ATG TGC | | | | |
| 38 | CATATG AAA AAG ACA | pET22b (+) ompASP₁₋₁₂- | + | + | + |
| | [symbol] | 6 × Arg-Xa-ofhepI** | | | |
| | [symbol] | | | | |
| | (ATC GAA GGT CGT) | | | | |
| | CAC ATC AGC CAC ATC | | | | |
| | TCC ATG TGC | | | | |
| 39 | CAT ATG AAA AAG ACA | pET22b (+) ompASP₁₋₁₄- | + | + | + |
| | GCT ATC GCG ATT | 6 × Arg-Xa-ofhepI** | | | |
| | [symbol] | | | | |
| | [symbol] | | | | |
| | CGT) CAC ATC AGC CAC | | | | |
| | ATC TCC ATG TGC | | | | |
| 40 | CAT ATG AAA AAG ACA | pET22b (+) ompASP₁₋₁₀- | + | +/− | +/− |
| | GCT ATC GCG ATT GCA | Xa-ofhepI** | | | |
| | GTG (ATC GAA GGT | | | | |
| | CGT) CAC ATC AGC CAC | | | | |
| | ATC TCC ATG TGC | | | | |
| 41 | CAT ATG AAA AAG ACA | pET22b (+) ompASP₁₋₁₀- | + | +/− | +/− |
| | GCT ATC GCG ATT GCA | LysArg-Xa-ofhepI** | | | |
| | [symbol] | | | | |
| | GGT CGT) CAC ATC AGC | | | | |
| | CAC ATC TCC ATG TGC | | | | |
| 42 | CAT ATG AAA AAG ACA | pET22b (+) ompASP₁₋₁₀- | + | + | + |
| | GCT ATC GCG ATT GCA | 4 × Arg-Xa-ofhepI** | | | |
| | [symbol] | | | | |
| | (ATC GAA GGT CGT) | | | | |
| | CAC ATC AGC CAC ATC | | | | |
| | TCC ATG TGC | | | | |
| 43 | CAT ATG AAA AAG ACA | pET22b (+) ompASP₁₋₁₀- | + | + | + |
| | GCT ATC GCG ATT GCA | 8 × Arg-Xa-ofhepI** | | | |
| | [symbol] | | | | |
| | [symbol] | | | | |
| | GAA GGT CGT) CAC ATC | | | | |
| | AGC CAC ATC TCC ATG | | | | |
| | TGC | | | | |
| 44 | CAT ATG AAA AAG ACA | pET22b (+) ompASP₁₋₁₀- | + | + | + |
| | GCT ATC GCG ATT GCA | 10 × Arg-Xa-ofhepI** | | | |
| | [symbol] | | | | |
| | [symbol] | | | | |
| | [symbol] | | | | |
| | CGT) CAC ATC AGC CAC | | | | |
| | ATC TCC ATG TGC | | | | |

Reverse primer

| | | |
|---|---|---|
| 45 | CTC GAG GTC GAC AAG | No corresponding clone |
| | CTT TTC GAA CTT GCA | |
| | GCA GGG GCC ACA GCC | |

CATwas extended to preserve an NdeI site.

Italic letters: indicate various sized oligonucleotides of OmpASP fragment.

Thick Italic letters: oligonucleotides of amino acids involved in pI and hydrophobicity/hydrophilicity average value.

Thick letters: oligonucleotides of hepcidin I.

ofhepI: ofHepcidin I gene.

Reverse primer: complementary oligonucleotide sequences to the sequence containing a C-terminal of ofHepcidin I and Glu/Hind III/Sal I/Xho I region.

Underlined thick letters: oligonucleotides of the factor Xa recognition site.

**: Glu/Hind III/Sal I/Xho I-6×His (Glu/Hind III/Sal I/Xho I derived from the reverse primer design and 6×His derived from His tag.)

Abbreviations: T-total protein; S-soluble fraction; and P-periplasm fraction.

Expression of recombinant of Hep I**: “−”; no-expression, “+/−”; weak expression, and “+”; expression.

TABLE 4
Hydrophobicity/hydrophilicity value of the signal sequence
of OmpASP₁₋₁₀-( )-Xa with the insertion of amino acids
having different pI and hydrophobicity/hydrophilicity
values in the ( ) region and the expression of soluble
olive flounder Hepcidin I in the clone of
pET22b(+)ompASP₁₋₁₀-( )-Xa-ofHepI** of FIG. 6 and Table 3

| | | | Hopp & | | | |
|---|---|---|---|---|---|---|
| | | pI | Woods scale | | Hopp & | Expression |
| | | value | hydrophobicity/ | | Woods scale | of |
| | | of the | hydrophilicity | Form | hydrophobicity/ | ofHepcid |
| | Inserted | inserted | of the | of | hydrophilicty of | in I in |
| | amino | amino | inserted | signal | the resulting | FIG. 6 and |
| | acid | acid | amino acid | peptide | signal peptide | Table 3 |
| Control | — | — | — | OmpASP₁₋₁₀- | −0.02 | +/− |
| | | | | ( )-Xa | | |
| 1 | 6 × Arg | 13.20 | 1.75 | OmpASP₁₋₁₀- | 0.88 | + |
| | | | | (6 × Arg)-Xa | | |
| 2 | 6 × Lys | 11.20 | 1.75 | OmpASP₁₋₁₀- | 0.88 | + |
| | | | | (6 × Lys)-Xa | | |
| 3 | 6 × Glu | 2.82 | 1.75 | OmpASP₁₋₁₀- | 0.88 | +/− |
| | | | | (6 × Glu)-Xa | | |
| 4 | 6 × Asp | 2.56 | 1.75 | OmpASP₁₋₁₀- | 0.88 | +/− |
| | | | | (6 × Asp)-Xa | | |
| 5 | 6 × Tyr | 5.55 | −1.33 | OmpASP₁₋₁₀- | −0.70 | +/− |
| | | | | (6 × Tyr)-Xa | | |
| 6 | 6 × Phe | 5.70 | −1.45 | OmpASP₁₋₁₀- | −0.76 | +/− |
| | | | | (6 × Phe)-Xa | | |
| 7 | 6 × Trp | 5.90 | −1.98 | OmpASP₁₋₁₀- | −1.03 | +/− |
| | | | | (6 × Trp)-Xa | | |

pI value and hydrophobicity/hydrophilicity (Hopp & Woods scale with window size: 6 and threshold line: 0.00) were calculated by DNASIS™. The ‘+value’ of Hopp and Woods scale hydrophobicity/hydrophilicity index indicates the inserted peptide is hydrophilic, whereas the ‘−value’ indicates hydrophobic. As absolute value increases, hydrophobicity/hydrophilicity increases. Expression of recombinant of Hep I**: “+/−”; weak expression, and “+”; expression.

### EXAMPLE 6

### Expression of Olive Flounder Hepcidin I According to the Change of Hydrophobicity/Hydrophilicity of a Signal Sequence

To investigate the expression of olive flounder Hepcidin I in relation with the hydrophobicity/hydrophilicity of the modified signal sequence, the present inventors examined the effect of the N-terminal of the OmpASP fragment acting as a directional signal. To do so, various OmpASP_{( )}-6×Arg-Xa with different lengths were designed and their corresponding clones were tested for expression. (Table 3 and FIG. 7). The Hopp & Woods hydrophobicity/hydrophilicity values of the modified signal sequences of OmpASP₁₋₆-6×Arg-Xa, OmpASP₁₋₈-6×Arg-Xa, OmpASP₁₋₁₀-6×Arg-Xa, OmpASP₁₋₁₂-6×Arg-Xa and OmpASP₁₋₁₄-6×Arg-Xa were 1.37, 1.09, 0.88, 0.69 and 0.62, respectively. The signal sequences having the Hopp and Woods scale hydrophilicity value of at lest 0.62 were all expressed in soluble form. The shorter the signal sequence, the higher the hydrophilicity and the more the expression in soluble form were observed. All of the sequences described above (OmpASP₁₆ through OmpASP₁₋₁₄) with average hydrophilicities of more than 0.62 directed the periplasmic expression of soluble recombinant Hepcidin I. As the length of the signal sequence decreased, the hydrophilicity increased, and the yield of soluble Hepcidin I increased. The shortest signal sequence (OmpASP₁₆; hydrophobicity −0.03) was linked with the 6×Arg-Xa sequence (hydrophilicity 1.47) to construct the resultant OmpASP₁₆-6×Arg-Xa (hydrophilicity 1.37), which showed an extended region of hydrophilicity in the hydropathy profile, lacking a hydrophobic curve at the N-terminus, whereas the other signal sequences (OmpASP₁₋₈, OmpASP₁₋₁₀, OmpASP₁₋₁₂, OmpASP₁₋₁₄) (hydrophobicity, see Table 2) were more hydrophobic than OmpASP₁₋₆, and the resultant signal sequences had asymmetrical hyperbolic curves of the typical transmembrane-like domain of the hydrophobic-hydrophilic curves in the profile. Therefore, it was suggested that the most preferable size of the signal sequence, in order to have transmembrane-like hydropathy exhibiting hydrophobic-hydrophilic curves, was at least OmpASP₁₋₈.

The present inventors also investigated the functions of the secretional enhancer in the C-terminal of the modified signal sequence. The signal sequence OmpASP₁₋₁₀ was set as a directional signal and OmpASP₁₋₁₀-( )-Xa was designed to include hydrophilic amino acids with different lengths in the -( )- region and the expression thereof was measured (Table 3 and FIG. 8). The Hopp & Wood scaled hydrophobicity/hydrophilicity values of the modified signal sequences of OmpASP₁₋₁₀-Xa, OmpASP₁₋₁₀-LysArg-Xa, OmpASP₁₋₁₀-4×Arg-Xa, OmpASP₁₋₁₀-6×Arg-Xa, OmpASP₁₋₁₀-8×Arg-Xa and OmpASP₁₋₁₀-10×Arg-Xa were −0.02, 0.35, 0.64, 0.88, 1.07 and 1.23, respectively. In conclusion, the signal sequences with Hopp & Woods scale hydrophilicity values ≦0.35 were too weak to direct the expression of soluble form, while the signal sequences with Hopp & Woods scale hydrophilicity values ≧0.64 were able to direct the expression of soluble form (FIG. 8). As the length of the hydrophilic amino acid was extended, the hydrophilicity and soluble expression were increased. The Hopp & Wood scale hydropathy profile of every signal sequence inducing soluble expression was further investigated. As a result, every signal sequence above had transmembrane-like hydropathy profile exhibited a hydrophobic curve in the N-terminal and a hydrophilic curve in the C-terminal.

It is judged from the above results that the hydrophobicity/hydrophilicity value of a signal sequence region determined by the Hopp & Woods scale can be a standard for a secretional enhancer for the soluble expression of olive flounder Hepcidin I and thereby the hydropathy profile according to the Hopp & Wood scale can be a secondary standard for a secretional enhancer.

### EXAMPLE 7

### The Relation Between the Hydropathy Profile According to the Hopp & Woods Scale of a Signal Sequence and the Expression of Olive Flounder Hepcidin I

It was proved in Example 6 that the Hopp & Woods scale hydrophobicity/hydrophilicity value was a reliable standard for the expression of olive flounder Hepcidin I in soluble form. Thus, the usability of the Hopp & Woods scale hydropathy profile as a standard for a secretional enhancer was investigated. The present inventors simulated the hydropathy profiles of the fusion protein of olive flounder Hepcidin I using ofHepcidin I as a control by computer program. ofHepcidinI, OmpASP₁₋₁₀-Xa-ofHepcidinI, OmpASP₁₋₁₀-LysArg-Xa-ofHepcidinI, and OmpASP₁₋₁₀-6×Arg-Xa-ofHepcidinI were investigated (FIG. 9). As a result, the simulated olive flounder Hepcidin I had an internal amphipathic domain, while the simulated OmpASP₁₋₁₀-Xa-ofHepcidinI and OmpASP₁₋₁₀-LysArg-ofHepcidinI had two transmembrane-like domains in similar sizes; one of which was originated from a signal sequence and the other was originated from the amphipathic domain of olive flounder Hepcidin I. The recombinant OmpASP₁₋₁₀-Xa-ofHepcidinI** and OmpASP₁₋₁₀-LysArg-ofHepcidinI** which were corresponding to the simulated OmpASP₁₋₁₀-Xa-ofHepcidinI and OmpASP₁₋₁₀-LysArg-ofHepcidinI fusion proteins were expressed in soluble form at a very low level (Table 3 and FIG. 8). However, the Hopp & Woods scale hydropathy profile of the simulated OmpASP₁₋₁₀-6×Arg-Xa-ofHepcidinI revealed that it had two transmembrane-like domains, one in the signal sequence and the other in the olive flounder Hepcidin I. The transmembrane-like domain in the signal sequence region was larger than the amphipathic domain in the olive flounder Hepcidin I. The corresponding clone produced a form of OmpASP₁₋₁₀-6×Arg-Xa-of HepcidinI** with enhanced solubility (FIG. 8) and the expression level was consistent with the size of transmembrane-like hydropathy profile.

Therefore, it is concluded that the expression of soluble target proteins in this system requires the leader sequence to have a hydropathy profile that corresponds to a transmembrane like domain that is larger than the amphipathic domain of the target protein.

The present inventors initially postulated that because olive flounder Hepcidin I had four disulfide bonds and an amphipathic domain, it would not be expressed as effectively as Mefp1 when fused with the OmpASP fragment. However, the above experiments suggested that a transmembrane-like domain would be the biggest barrier. The disulfide bonds are formed when the nascent polypeptide chains are secreted to the periplasm, on oxidizing environment where disulfide isomerases such as DsbA are present (Bardwell et al., Cell 67, 581-589, 1991; Kamitani et al., EMBO J. 11, 57-62, 1992). Co-expression of DsbA as a potential folding aid does not influence the yield of an active target protein (Beck and Burtscher, Protein Expr. Purif. 5, 192-197, 1994). Therefore, the inventors postulate that the nascent Hepcidin I polypeptide is secreted to the periplasm without forming any disulfide bonds or at least it does not encounter any structural obstacle caused by disulfide bonds.

### INDUSTRIAL APPLICABILITY

As explained hereinbefore, the method of the present invention is effectively used for the production of a recombinant heterologous protein by preventing the generation of an insoluble precipitate and improving the secretional efficiency to the periplasm. In addition, the method of the invention can be effectively used for the transduction of a therapeutic protein by increasing the membrane permeability by hiring a strong secretional enhancer.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An expression vector for increasing secretional efficiency of a heterologous protein, comprising a gene construct composed of:
(i) a promoter; and
(ii) a polynucleotide encoding a polypeptide fragment comprising a region of a signal sequence operably linked to the promoter.

2. The expression vector according to claim 1, wherein the promoter is a viral promoter, a prokaryotic promoter or a eukaryotic promoter

3. The expression vector according to claim 2, wherein the viral promoter is selected from a group consisting of: a cytomegalovirus (CMV) promoter, a polyomavirus promoter, a fowl pox virus promoter, an adenovirus promoter, a bovine papillomavirus promoter, a rous sarcomavirus promoter, a retrovirus promoter, a hepatitis B virus promoter, a herpes simplex virus thymidine kinase promoter and a simian virus 40 (SV40) promoter.

4. The expression vector according to claim 2, wherein the prokaryotic promoter is selected from a group consisting of: a T7 promoter, a SP6 promoter, a heat-shock protein 70 promoter, a β-lactamase, a lactose promoter, an alkaline phosphatase promoter, a tryptophane promoter and a tac promoter.

5. The expression vector according to claim 2, wherein the eukaryotic promoter is a yeast promoter, a plant promoter or an animal promoter.

6. The expression vector according to claim 5, wherein the yeast promoter is selected from a group consisting of: a 3-phosphoglycerate kinase promoter, an enolase promoter, a glyceraldehyde-3-phosphate dehydrogenase promoter, a hexokinase promoter, a pyruvate dicarboxylase promoter, a phosphofructokinase promoter, a glucose-6-phosphate isomerase promoter, a 3-phosphoglycerate mutase promoter, a pyruvate kinase promoter, a triosphosphate isomerase promoter, a phosphoglucose isomerase promoter, a glucokinase promoter, an alcohol dehydrogenase 2 promoter, an isocytochrome C promoter, an acidic phosphatase promoter, a Saccharomyces cerevisiae GAL1 promoter, a Saccharomyces cerevisiae GAL7 promoter, a Saccharomyces cerevisiae GAL10 promoter and a Pichia pastoris AOX1 promoter.

7. The expression vector according to claim 5, wherein the animal promoter is selected from a group consisting of a heat-shock protein promoter, a proactin promoter and an immunoglobulin promoter.

8. The expression vector according to claim 1, wherein the signal sequence is a viral signal sequence, a prokaryotic signal sequence or a eukaryotic signal sequence or leader sequence.

9. The expression vector according to claim 1, wherein the signal sequence is selected from a group consisting of: an OmpA signal sequence, a CT-B (cholera toxin subunit B) signal sequence, a LTIIb-B (E. coli heat-labile enterotoxin B subunit) signal sequence, a BAP (bacterial alkaline phosphatase) signal sequence, a yeast carboxypeptidase Y signal sequence, a Kluyveromyces lactis killer toxin gamma subunit signal sequence, a bovine growth hormone signal sequence, an influenza neuraminidase signal-anchor, a translocon-associated protein subunit alpha signal sequence and a Twin-arginine translocation (Tat) signal sequence.

10. The expression vector according to claim 1, wherein the polypeptide fragment the N-region is peptide composed of 3-21 amino acids rising the 1^{st}-the 3^{rd} amino acids of the signal sequence.

11. The expression vector according to claim 1, wherein the pI value of the polypeptide fragment comprising the N-region is at least 8.

12. The expression vector according to claim 1, wherein the polynucleotide encoding the polypeptide fragment comprising the N-region additionally contains an operably linked secretional enhancer.

13. The expression vector according to claim 12, wherein the secretional enhancer is a polynucleotide encoding a hydrophilic peptide composed of 2-50 amino acids among which at least 60% are hydrophilic amino acids.

14. The expression vector according to claim 1, wherein the nucleotide encoding a protease recognition site operably linked to the nucleotide encoding a polypeptide containing the N-region is additionally included.

15. The expression vector according to claim 14, wherein the protease recognition site is selected from a group consisting of: a factor Xa recognition site, an enterokinase recognition site, a genenase I recognition site and a furin recognition site independently or in fusion forms.

16. The expression vector according to claim 12, wherein the nucleotide encoding the secretional enhancer is operably linked to nucleotide encoding a protease recognition site.

17. The expression vector according to claim 16, wherein the protease recognition site is selected from a group consisting of: a factor Xa protease recognition site, an enterokinase recognition site, a genenase I recognition site and a furin recognition site independently or in fusion forms.

18. The expression vector according to claim 1, wherein a restriction enzyme site is additionally included for the introduction of a gene encoding a heterologous protein.

19. The expression vector according to claim 18, wherein the heterologous protein does not have one or more of a transmembrane domain, a transmembrane-like domain or an amphipathic domain.

20. The expression vector according to claim 18, wherein the heterologous protein is Mefp1 without an internal transmembrane domain, a transmembrane-like domain or an amphipathic domain.

21. The expression vector according to claim 1, wherein the gene construct is operably linked to polynucleotide encoding a heterologous protein.

22. An expression vector for improving secretional efficiency of a heterologous protein, comprising a gene construct composed of:
(i) a promoter,
(ii) a polynucleotide encoding a hydrophobic fragment comprising a N-region and central characteristic hydrophobic region of a signal sequence operably linked to the promoter, and
(iii) a secretional enhancer operably linked to the polynucleotide.

23. The expression vector according to claim 22, wherein the promoter is a viral promoter, a prokaryotic promoter or a eukaryotic promoter.

24. The expression vector according to claim 23, wherein the viral promoter is selected from a group consisting of: a cytomegalovirus (CMV) promoter, a polyomavirus promoter, a fowl pox virus promoter, an adenovirus promoter, a bovine papillomavirus promoter, a rous sarcomavirus promoter, a retrovirus promoter, a hepatitis B virus promoter, a herpes simplex virus thymidine kinase promoter and a simian virus 40 (SV40) promoter.

25. The expression vector according to claim 23, wherein the prokaryotic promoter is selected from a group consisting of: a T7 promoter, a SP6 promoter, a heat-shock protein 70 promoter, a β-lactamase, a lactose promoter, an alkaline phosphatase promoter, a tryptophane promoter and a tac promoter.

26. The expression vector according to claim 23, wherein the eukaryotic promoter is a yeast promoter, a plant promoter or an animal promoter.

27. The expression vector according to claim 26, wherein the yeast promoter is selected from a group consisting of: a 3-phosphoglycerate kinase promoter, an enolase promoter, a glyceraldehyde-3-phosphate dehydrogenase promoter, a hexokinase promoter, a pyruvate dicarboxylase promoter, a phosphofructokinase promoter, a glucose-6-phosphate isomerase promoter, a 3-phosphoglycerate mutase promoter, a pyruvate kinase promoter, a triosphosphate isomerase promoter, a phosphoglucose isomerase promoter, a glucokinase promoter, an alcohol dehydrogenase 2 promoter, an isocytochrome C promoter, an acidic phosphatase promoter, a Saccharomyces cerevisiae GAL1 promoter, a Saccharomyces cerevisiae GAL7 promoter, a Saccharomyces cerevisiae GAL10 promoter and a Pichia pastoris AOX1 promoter.

28. The expression vector according to claim 26, wherein the animal promoter is selected from a group consisting of: a heat-shock protein promoter, a proactin promoter and an immunoglobulin promoter.

29. The expression vector according to claim 22, wherein the signal sequence is a viral signal sequence, a prokaryotic signal sequence or a eukaryotic signal sequence or leader sequence.

30. The expression vector according to claim 22, wherein the signal sequence is selected from a group consisting of: an OmpA signal sequence, a CT-B (cholera toxin subunit B) signal sequence, a LTIIb-B (E. coli heat-labile enterotoxin B subunit) signal sequence, a BAP (bacterial alkaline phosphatase) signal sequence, a yeast carboxypeptidase Y signal sequence, a Kluyveromyces lactis killer toxin gamma subunit signal sequence, a bovine growth hormone signal sequence, an influenza neuraminidase signal-anchor, a translocon-associated protein subunit alpha signal sequence and a Twin-arginine translocation (Tat) signal sequence.

31. The expression vector according to claim 22, wherein the hydrophobic fragment of the signal sequence is a peptide composed of 6-21 amino acids comprising the 1^{st}-the 6^{th} amino acids of the signal sequence.

32. The expression vector according to claim 22, wherein the secretional enhancer is a polynucleotide encoding a peptide composed of 2-50 amino acids among which at least 60% are hydrophilic amino acids.

33. The expression vector according to claim 22, wherein the secretional enhancer is a polynucleotide encoding a hydrophilic peptide having pI value of at least 10.

34. The expression vector according to claim 32, wherein the hydrophilic amino acid is lysine or arginine.

35. The expression vector according to claim 22, wherein the secretional enhancer is a polynucleotide encoding a peptide having the repeat of 6 hydrophilic amino acids.

36. The expression vector according to claim 22, wherein the polynucleotide encoding a protease recognition site is additionally operably linked to the polynucleotide encoding the secretional enhancer.

37. The expression vector according to claim 22, wherein the restriction enzyme site for the insertion of a foreign gene is additionally linked to the polynucleotide encoding a secretional enhancer.

38. The expression vector according to claim 22, wherein the polynucleotide encoding the heterologous protein is additionally operably linked to the gene construct.

39. The expression vector according to claim 37, wherein the heterologous protein has one or more internal transmembrane domains, transmembrane-like domains or amphipathic domains.

40. The expression vector according to claim 39, wherein the heterologous protein is olive flounder Hepcidin I.

41. A non-human transformant prepared by transforming a host cell with the expression vector of claim 1.

42. A method for improving secretional efficiency of a heterologous protein comprising:
1) analyzing hydropathy profile of a heterologous protein;
2) judging whether the heterologous protein analyzed in 1) contains one or more of a transmembrane domain, a transmembrane-like domain or an amphipathic domain inside;
3) (a) constructing a gene construct composed of polynucleotides encoding a fusion protein in which the heterologous protein is combined with a polypeptide fragment containing a N-region of a signal sequence or a fusion protein in which the heterologous protein is combined with a polypeptide fragment containing the N-region of a signal sequence and a protease recognition site, when the heterologous protein is confirmed not to contain a transmembrane domain, transmembrane-like domain or amphipathic domain in 2), and
(b) constructing a gene construct composed of polynucleotides encoding a fusion protein containing a hydrophobic fragment comprising the N-region and central characteristic hydrophobic region of a signal sequence, a secretional enhancer and the heterologous protein sequentially or a fusion protein containing a hydrophobic fragment comprising the N-region and central characteristic hydrophobic region of a signal sequence, a secretional enhancer, a protease recognition site and the heterologous protein sequentially, when the heterologous protein is confirmed to have one or more of a transmembrane domain, a transmembrane-like domain and an amphipathic domain in 2);
4) constructing a recombinant expression vector by inserting the gene construct prepared in 3) operably into an expression vector;
5) constructing a transformant by transforming a host cell with the recombinant expression vector of 4); and
6) culturing the transformant of 5).

43. The method according to claim 42, wherein the heterologous protein is an insoluble protein.

44. The method according to claim 42, wherein the hydropathy profile is analyzed by computer software or a web-based application for hydropathy profile analysis.

45. The method according to claim 44, wherein the computer software is selected from a group consisting of DNASIS™, Visual OMP, Lasergene, pDRAW32 and NetSupport.

46. The method according to claim 42, wherein the secretional enhancer is a polypeptide composed of 2-50 amino acids among which at least 60% are hydrophilic amino acids.

47. The method according to claim 42, wherein the secretional enhancer is a hydrophilic peptide having pI value of at least 10.

48. The method according to claim 46, wherein the hydrophilic amino acid is lysine or arginine.

49. The method of claim 42 further comprising
7) separating a fusion heterologous protein from the culture solution of 6).

50. The method of claim 49 further comprising
8) separating the native form of the heterologous protein from the fusion protein separated in 7) after digesting the protease recognition site with a protease.

51. A method for improving secretional efficiency of a heterologous protein comprising:
1) constructing a recombinant expression vector by operably linking a polynucleotide encoding a heterologous protein to the restriction enzyme site of the expression vector of claim 18;
2) generating a transformant by transforming a host cell with the recombinant expression vector of 1); and
3) culturing the transformant of 2).

52. A method for improving secretional efficiency of a heterologous protein comprising:
1) constructing a recombinant expression vector by operably linking a gene encoding a heterologous protein to the restriction enzyme site of the expression vector of claim 37;
2) generating a transformant by transforming a host cell with the recombinant expression vector of 1); and
3) culturing the transformant of 2).

53. A method for preparing the native form of a heterologous protein comprising:
1) generating a transformant by transforming a host cell with the expression vector of claim 38;
2) culturing the transformant of 1);
3) separating the heterologous protein from the culture solution; and
4) separating the native form of the heterologous protein by treating a protease to the separated heterologous protein.

54. The method according to claim 52, wherein the heterologous protein is a therapeutic protein targeting the brain.

55. A recombinant heterologous protein, which is prepared by the method of claim 54, and has a transmembrane region facilitating the passing through blood-brain barrier.

56. A pharmaceutical composition containing the protein of claim 55 and a pharmaceutically acceptable carrier.

57. The pharmaceutical composition according to claim 56, which is used for the treatment of brain disease.

58. The transformant according to claim 41, wherein the host cell is a prokaryotic cell or a eukaryotic cell.

59. The transformant according to claim 58, wherein the prokaryotic cell is selected from a group consisting of virus, E. coli and Bacillus.

60. The transformant according to claim 58, wherein the eukaryotic cell is selected from a group consisting of mammalian cells, insect cells, yeasts and plant cells.

61. A screening method for a secretional enhancer improving secretional efficiency of a heterologous protein, which comprises:
1) constructing an expression vector containing a gene construct in which a promoter, a polynucleotide encoding a polypeptide fragment containing the N-region of a signal sequence or a hydrophobic fragment containing the N-region and central characteristic hydrophobic region of a signal sequence, a restriction enzyme site for the insertion of a secretional enhancer candidate and a polynucleotide encoding a heterologous protein are operably linked to one another;
2) constructing a recombinant expression vector by inserting a polynucleotide encoding a secretional enhancer candidate sequence comprising hydrophilic amino acids into the restriction enzyme site of the expression vector;
3) generating a transformant by transforming a host cell with the recombinant expression vector of 2);
4) Culturing the transformant of 3);
5) measuring the expression level of the heterologous protein in culture solutions of both the transformant (control) transformed with the expression vector of 1) and the transformant of 4); and
6) selecting a secretional enhancer which significantly increases the expression level of the heterologous protein inserted, compared with a control.

62. The expression vector according to claim 12, wherein a restriction enzyme site is additionally included for the introduction of a gene encoding a heterologous protein.

63. A non-human transformant prepared by transforming a host cell with the expression vector of claim 22.

64. The expression vector according to claim 38, wherein the heterologous protein is a protein having one or more internal transmembrane domains, transmembrane-like domains or amphipathic domains.
